(19) United States
(12) Patent Application Publication
(10) Pub. No.: US 2024/0240149 A1
(43) Pub. Date: Jul. 18, 2024

(54) A METHOD FOR PRODUCING BLOOD PROGENITOR AND PROGENITOR T CELLS, RESULTING CELLS AND METHODS AND USES THEREOF

(71) Applicant: The University of British Columbia, Vancouver (CA)

(72) Inventors: Peter W. Zandstra, Vancouver (CA); Yale S. Michaels, Vancouver (CA)

(21) Appl. No.: 18/561,873

(22) PCT Filed: May 18, 2022
(86) PCT No.: PCT/CA2022/050789

§ 371 Date: Nov. 17, 2023

Related U.S. Application Data
(60) Provisional application No. 63/276,887, filed on Nov. 8, 2021.
(60) Provisional application No. 63/190,037, filed on May 18, 2021.

(51) Int. Cl. C12N 5/0789

(52) U.S. Cl. CPC C12N 5/0647; C12N 2500/24; C12N 2500/38; C12N 2500/44; C12N 2501/105; C12N 2501/115; C12N 2501/125; C12N 2501/155; C12N 2501/165; C12N 2501/2303; C12N 2501/2306; C12N 2501/2311; C12N 2501/26; C12N 2501/42; C12N 2501/585; C12N 2506/28; C12N 2513/00

(57) ABSTRACT

Described herein is a method for producing blood progenitor (hematopoietic progenitor cells) and T cell progenitor cells and to cells produced or obtainable by the process and the use of said cells, the method including: (a) optionally subjecting pluripotent stem cells under conditions that direct the cells to become mesoderm and subsequently hemogenic endothelial cells; and (b) directing hemogenic endothelial cells to differentiate into blood progenitor cells, preferably defined blood progenitor cells) using a media formulation designed to promote endothelial to hematopoietic transition (EHT) while being cultured on a surface functionalised with ligands designed to activate the Notch signaling pathway. In some aspects the ligands are Notch ligands, such as DLL4 and integrin ligands, such as integrin α4β1 ligand or VCAM1.

### RELATED APPLICATIONS

This application is related and claims priority from U.S. Provisional Patent Application No. 63/190,037, filed May 18, 2021, and U.S. Provisional Patent Application No. 63/276,887, filed Nov. 8, 2021, both of which are incorporated herein by reference.

### FIELD OF THE INVENTION

The present invention relates to the field of a method for enhancing the production of blood progenitor cells and progenitor T cells, and to the resulting cells and cell populations. In some aspects the process for producing the cells comprises an endothelial to hematopoietic transition (EHT) step. In some other aspects the method comprises culturing hemogenic endothelial cells under conditions that promote EHT while enhancing the activation and/or activating the Notch signaling pathway. In yet some other aspects, the invention provides kits, methods and uses of the foregoing.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “SequenceListing_T8483514WO.txt”, which was created on May 18, 2022 and is 19,238 bytes in size. The subject matter of the Sequence Listing is incorporated herein in its entirety by reference.

The sequence listing in this description is also being provided in computer readable form. It is hereby stated that the information recorded in computer readable form is identical to the written sequence listing in this description.

### BACKGROUND OF THE INVENTION

Pluripotent stem cells (PSCs) are cells that can self-renew. Self-renewal is the capacity of the stem cells to divide indefinitely, producing unaltered cell daughters maintaining the same properties of the progenitor cell. In particular conditions or under specific signals, a stem cell is able to exit from self-renewal and engage a program leading to differentiation into specialized cell types, through various progenitor cells, such as blood progenitor cells and progenitor T cells.

The importance of embryonic stem cells (ESCs) and induced PSCs (iPSCs), such as human ESCs and iPSCs, as a potentially unlimited source of various differentiated cell types, such as blood progenitor cells and progenitor T cells and potentially mature cells for therapy, such as regenerative and immunotherapies has long been recognized but obtaining a consistent viable source suitable for human therapies and other uses has been challenging.

In the hematopoietic system, self-renewal capacity is the privilege of rare multipotent cells named hematopoietic stem cells (HSCs). Their closest progeny, hematopoietic progenitor cells (HPCs), may be multipotent, oligopotent or unipotent. While HPCs lack significant self-renewing capacity, they are capable of further differentiation into mature blood cells of all hematopoietic lineages. HSCs are responsible for the development, maintenance and regeneration of all blood forming tissues in the body. They are also critical for long-term engraftment and reconstitution in the setting of bone marrow transplantation (BMT). Many applications of HSC and HPCs do not differentate between the utility of these two populations of cells, for example in studies that address trafficking cell populations enriched in both stem and progenitor cells, i.e., combined population of hematopoietic stem/progenitor cells (HSPC) and analyzed. (Mazo et al, Trends Immunol. 2011 October, 32(10): 493-503).

The molecular and cellular signals that guide T cell development from hematopoietic stem cells (HSCs) and hematopoietic progenitor cells (HPCs), remain poorly understood. The thymic microenvironment integrates multiple niche molecules to potentiate T cell development in vivo. Traditional methods have used feeder cell or animal products to maintain and differentiate cell cultures, which is not suitable to satisfy various safety standards for biological ingredients.

There are existing methods for taking pluripotent stem cells and converting them into HSC-like cells and then T cells but each of these existing methods suffers from one or more of the following limitations, including:
(a) they require co-culture with immortalised feeder cell lines and/or undefined serum. These are variable and difficult to translate to the clinic. These methods are also a black box because it is not known what the feeder cells are producing, or what is in the serum. Further, it is difficult to modify and improve these protocols;
(b) they are inefficient at producing late stage or mature T cells;
(c) they produce cells at very low yields or cells that are limited in their development and maturation.

For instance, U.S. Pat. No. 10,858,628, issued Dec. 8, 2020 (Valamehr et al.) make HSC-like cells using an environment that comprises Matrigel, a protein mixture secreted by mouse sarcoma cells. Further, they produce very low levels of late stage progenitor T cells. In one embodiment, they suggest using plate bound DLL4 to make progenitor T cells but they only detect fairly early T cell progenitors with this method (CD7+) and not more developed CD4+, CD8+ progenitors or more mature CD8+ T cells.

In WO/2021/032855 A1 published Feb. 25, 2021 (Yang Cheng Tao et al), describes a method for producing HSC-like cells from hemogenic endothelial cells but the CD4+, CD8+, CD3+ T cell output appears to be very low (<10%).

Others, such as Uenishi et al (Nature Communications (2018) 9:1828) and Kumar et al (J Immunol 2019; 202:770-776 (pre-published on line Dec. 21, 2018)); and Montel-Hagen et al (Cell Cell Stem Cell 24, 376-389, Mar. 7, 2019^{a} 2018 Elsevier Inc.) use immortalised feeder cell lines to generate HSC-like cells and T cells, which has the issues as noted above.

There remains an important unmet need to optimize the differentiation of pluripotent stem cells into progenitor cells, including blood and T cell progenitor cells in a manner that produces desired cell types in a consistent manner suitable for various uses, such as various therapies, such as regenerative and immunotherapies. In other aspects, there is a need to be able to produce high yields of the desired cell types. In other embodiments, there is a need for the process to be a feeder-free or non-xenogenic process, and/or process that does not use undefined serums or animal-derived products. So in one aspect there is a need for an in vitro, stromal cell-free system.

### SUMMARY OF THE INVENTION

In some aspects, the present invention produces and enhances the generation and/or production of blood progenitor cells (hematopoietic stem and hematopoietic progenitor cells or HSPCs), including lymphoid-competent progenitors, from hemogenic endothelial cells wherein the hemogenic endothelial cells are cultured under conditions that activate, commit and direct the cells and/or promote endothelial to hematopoietic transition (EHT) while activating and/or enhancing the activation of the Notch signalling pathway. Wherein in some embodiments, activating and/or enhancing the activation of the Notch signalling pathway comprises culturing the cells on a surface functionalised with ligands that activate and/or enhance the activation of the Notch signaling pathway. In some further embodiments that ligands are a Notch ligand (e.g. DLL4) and an integrin ligand, such as an integrin α4β1 ligand, or such as VCAM1. The methods of the invention enhance the transition from endothelial to hematopoietic cell fates during an early stage of differentiation.

In some further aspects of the invention the blood progenitor cells produced are definitive blood progenitor cells. In some other embodiments of the invention the blood progenitor cells express a molecular signature. In some embodiments the molecular signature comprises expression of both HLF and HOXA9 and optionally one or both of RAB27B and IFGBP2. In some further embodiments, the blood progenitor cells have multilineage developmental capacity. In some other embodiments the blood progenitor cells can develop into differentiated blood cells. In some embodiments the differentiated blood cells are selected from the group consisting of red blood cells, T-cells, B-cells, macrophages, erythrocytes, megakaryocytes, granulocytes, neutrophils, natural killer (NK) cells, mast cells and eosinophils.

In some further embodiments of the invention, the methods comprise producing and/or enhancing the generation and/or production of blood progenitor cells from pluripotent stem cells (PSCs), the method comprising a blood induction step to generate hemogenic endothelial cells from the pluripotent stem cells, which are then committed and directed to form blood progenitor cells via the EHT step method noted above. In a further, embodiment, the resulting blood progenitor cells can be cultured under conditions that promote lymphoid specification (lymphopoiesis), differentiation into progenitor T cells and T cell differentiation. In some aspects said conditions that promote lymphoid specification (lymphopoiesis), differentiation into progenitor T cells and T cell differentiation comprises activating and/or enhancing the activation of the Notch signalling pathway. Wherein in some embodiments, activating and/or enhancing the activation of the Notch signalling pathway comprises culturing the cells on a surface functionalised with ligands that activate and/or enhance the activation of the Notch signaling pathway. In some further embodiments that ligands are a Notch ligand (e.g. DLL4) and an integrin ligand, such as an integrin α4β1 ligand, or such as VCAM1. In some aspects, the invention provides methods for enhancing the production of blood progenitor cells and/or cells derived from blood progenitor cells such as progenitor Tcells, early stage T cell progenitor cells, later stage progenitor T cells and mature T cells with high efficiency.

In some aspects the cells produced from the hemogenic endothelial cells (i.e. from the hemogenic endothelium) are definitive blood progenitor cells (definitive hematopoietic stem and progenitor cells).

In some other aspects, the cells produced are definitive blood progenitor cells expressing a molecular signature. In some embodiments the molecular signature of the blood progenitor cells is the expression of both HLF and HOXA9 and optionally one or both of RAB27B, and IGFBP2

In some other aspects of the invention, the blood progenitor cells produced by the method(s) of the invention have multilineage developmental capacity.

In yet some other embodiments, the blood progenitor cells produced by the methods of the invention can develop into differentiated blood cells including red blood cells, T cells, B cells, macrophages, erythrocytes, megakaryocytes, granulocytes, neutrophils, natural killer (NK) cells, mast cells, eosinophils (e.g. 2018 Cell Stem Cell Dzierzak, FIG. 2)

In some aspects, the present invention provides a method that better optimizes the differentiation of pluripotent stem cells into progenitor cells, including blood and T cell progenitor cells in a manner that produces desired cell types in a consistent manner suitable for various uses, such as various therapies, such as regenerative and immunotherapies. In other aspects, the invention provides a process that results in high or higher yields of the desired cell types. In other aspects, the methods and processes of the invention are feeder-free, and/or serum-free and/or non-xenogenic and/or an in vitro stromal cell-free system, and/or process that does not use undefined serums or animal-derived products. As such, the invention, in some aspects, provides methods and processes using defined culture media.

The present invention relates to the field of a method for producing blood progenitor cells and progenitor T cells from pluripotent stem cells (PSCs). In one aspect the method of the invention comprises committing the cells to an endothelium-hematopoietic transition also interchangeably referred to an endothelial-hematopoetic transition (EHT) to produce blood progenitor cells. In some aspects of the invention the resulting blood progenitor cells are where those cells are uniquely capable of lymphoid specification (lymphopoiesis) and producing progenitor T cells with special properties.

In other aspects, the invention provides a novel use in the EHT step of the process of adsorbed or immobilized notch signaling ligands and integrin ligands. In some embodiments the integrin ligand is a vascular cell adhesion molecule, such as vascular cell adhesion molecule 1—also known as vascular cell adhesion protein 1) and in other aspects to media comprising said ligands. In other aspects of the invention said adsorbed or immobilized ligands can also be used during the blood progenitor to progenitor T cell generation step of the process.

In some aspects the invention provides one or more of the components that can be used in the methods of the invention, such as the starting components (such as the pluripotent stem cells or other cells, culture media, and/or funcationalised surfaces, and/or the ligands; and/or any one or more of the generated components (such as the blood progenitor cells or other cells derived from the methods of the invention). In some embodiments the invention provides cell culture media and/or compositions that can be used in one or more of the steps and/or stages of the methods of the invention. In some aspects the invention provides the surface and or functionalized surface, with or without the requisite ligands that can be used in the methods of the invention. In other aspects, the invention provides directions for use of said one or more components in the methods of the invention. In some aspects, the inventions provides kits comprising one or more of said components to carry out the method(s) and/or one or more of the steps of the method(s) of the invention and optionally instructions for use.

In some other aspects, the invention provides cells that are the products of the process, such as novel isolated blood progenitor cells and isolated progenitor T cells. In yet some other aspects the invention provides isolated cells (or cell populations) with unique gene expression profiles. In some other aspects, the invention provides cells that are derived from the blood progenitor cells produced by the method(s) of the invention.

In some other aspects, the invention provides methods and uses of the foregoing.

The present invention provides a process or method for producing pluripotent stem cells (PSCs) derived cells (such as human PSCs derived cells, such as hematopoietic and T cell progenitor cells. In some aspects the process is a feeder-free process, and/or serum-free, and/or non-xenogenic process and/or process that does not use serums or undefined serums or animal-derived products, such as an in ex vivo system and/or an in vitro, stromal cell-free system.

In some aspects, the present invention uses a culture stage where media is applied that supports endothelial to hematopoietic transition (EHT) in the presence of immobilized or adsorbed signaling ligands, such as notch signaling ligands (notch ligand), such as the recombinant proteins DLL4, and integrin ligands, such as VCAM1 (which is a cell adhesion molecule and also an integrin ligand). In some aspects, the immobilization or adsorbed ligands is on functionalized substrate(s). In some aspects the ligands, such as the Notch signalling ligand (e.g. DLLR) and the integrin ligand (e.g. VCAM-1) have synergistic effect in the production of the desired cell types, such as the blood progenitor cells and progenitor T cells.

The cells that come out of this culture stage can then be committed to becoming T cells (including but not necessarily limited to early stage T cell progenitor cells, later stage progenitor T cells and mature T cells) with high efficiency.

In another aspect of the invention, the recombinant proteins, the Notch and integrin ligands, such as DLL4 and VCAM-1 are adsorbed or immobilized on a surface. In another aspect the process does not require feeder cells and is non-xenogenic.

In other aspects the methods and processes of the present invention produce higher levels of mature T cell progenitors (e.g., in some embodiments, >70%).

In some aspects the method of the present invention produces not only fairly early T cell progenitors (CD7+), but later stage, more developed T cells can be obtained (e.g., CD4+, CD8+ progenitors and CD8+ mature T cells). In some embodiments of the invention, the methods of the invention result in a diverse population of such T cells and T cell progenitors, for instance as indicated by multiple different recombined TCR sequences. In some aspects the population of such T cells and/or T cell progenitor cells generated by the method of the present invention have similar diversity as naturally occurring T cell populations in vivo.

In some embodiments, the present invention provides a method for improving and enhancing T cell production from PSCs by using a three-step (or phase) process by promoting differentiation of PSCs through an endothelial-to-hematopoietic transition (EHT) phase to produce blood progenitor cells as opposed to directly to a T cell production phase. In some aspects, the method enhances the number of cells that go through the EHT in the process of producing blood progenitor cells.

In some aspects the three phases can be described as the blood induction phase, the EHT phase and the T cell differentiation, specification or production phase. The process of the present invention, in addition to using chemically defined media to promote the endothelial-to-hematopoietic transition, promotes Notch signaling during EHT. In some embodiments Notch signaling is promoted using a combination of a notch signaling ligand and an integrin ligand. In some other embodiments Notch signaling is promoted using the combination of a Notch ligand, such as DLL4 and an integrin ligand such as VCAM1. In some aspects the invention provides a substantial benefit in the production of T cells (progenitors, early stage and later stage T cells), in yield and type of T cell or T cell progenitor produced and in the diversity of the T cell population produced.

Both the blood progenitors (or hematopoietic cells) and T cells produced or capable of being produced by the method(s) of the invention have unique transcriptional profiles and are different transcriptional profiles than cells produced by conventional methods, and each have various therapeutic uses. They can be isolated and thus the present invention, in some aspects, provides isolated blood progenitor and T cells produced or obtainable by the methods of the present invention.

In some aspects, the blood progenitor or hematopoietic cells produced by the invention express genes that are expressed by primary human hematopoietic stem cells including SPN, PTPRC, HLF and THY1. In some aspects, the hematopoietic cells produced by the invention can differentiate to become myeloid progenitors, mast cell progenitors, lymphoid progenitors and erythroid progenitors that express foetal and adult hemoglobin genes including HBG2 and HBB.

In some other aspects, the progenitor T cells produced by the invention express CD7, IL7R, PTCRA as well as high levels of IGLL1, SRGN and CXCR4. In some other aspects, the progenitor T cells produced by the invention progress through a highly proliferative stage followed by a non-cycling stage. In yet other aspects of the invention, the progenitor T cells produced by the invention express high levels of class-I HLA genes including HLA-A and HLA-B and B2M. The T cell progenitors produced by the method of the present invention when using unbiased leiden clustering, cluster separately from those produced in the prior art 2 step process.

Thus, in one aspect the present invention provides a chemically defined platform for producing mature T cells from PSCs and/or hemogenic endothelial cells.

In another aspect, the invention provides a method for producing progenitor T cells comprising: (a) subjecting pluripotent stem cells to staged media formulations that direct the cells to become mesoderm and subsequently hemogenic endothelial cells wherein in some preferred embodiments the pluripotent stem cells are aggregated pluripotent stem cells. In some embodiments the aggregated pluripotent stem cells are aggregated into 3-dimensional multi-cellular structures, such as by centrifugation in microwell plates, partial dissociation from adherent cell cultures, dissociation to single cells followed by culture on non-adherent surfaces, or by other methods; (b)(i) dissociating the aggregated cells and optionally enriching the CD34+ population which contains within it hemogenic endothelial cells, and (ii) directing the hemogenic endothelial cells to differentiate into blood progenitor cells using a media formulation designed to promote endothelial to hematopoietic transition (EHT) while being cultured on a surface functionalised with ligands designed to activate (including promoting or enhancing the activation of) the Notch signaling pathway (such as Notch ligand, such as DLL4 and integrin ligand, such as a vascular cell adhesion ligand, such as VCAM-1; and (c) culturing the blood progenitor cells in media designed to promote lymphoid specification (lymphopoiesis or directed to become lymphoid progenitors), differentiation into progenitor T cells and T cell differentiation. In some aspects, the media and culture conditions to promote lymphoid specification and differentiation into progenitor T cells and T cell differentiation comprises culturing the blood progenitor cells on a surface functionalised with ligands that activate or enhance activation of T cell development, such as, in some aspects ligands that that activate and/or enhance activation of the Notch signaling pathway. It should be understood a person of skill in the art, that invention also provides a method of producing and enhancing the production of blood progenitor cells using the method of step (b)(ii) above and/or producing and/or enhancing the production of the progenitor T cells commencing at step (b)(ii) above, with the steps (a) and (b)(i) being optional. In some embodiments of the invention aggregating the pluripotent stem cells is a preferred method. In other embodiments dissociating the aggregated cells and/or enriching the CD34+ cell population which comprises hemogenic endothelial cells is a preferred method.

The cells produced by the method(s) of the present invention can then be used to produce and/or enhance the production of cells derived from the blood progenitor cells and progenitor T cells generated by the methods of the invention.

In some aspects of the method the ligands used in the EHT step of the process step (b) above) and the ligands used to activate the T cell competent blood progenitor cell (step (c) above) are the same. In some aspects they are a Notch ligand (such as Delta-like 4 (DLL4)) and an integrin ligand (such as VCAM-1).

In some preferred embodiments of the invention, the ligands used in the methods of the invention (e.g. steps (b) and (c) above are adsorbed or immobilized on a surface functionalized with said ligands. In some aspects the surface is a manufactured (or human made) surface. In some aspects, the surface surface functionalised with ligands is selected from the group consisting of: a two dimensional tissue culture surface; a tissue culture plate; the surface of beads; the surface of hydrogels; and other suitable surfaces. In some other embodiments these molecules or ligands used in the methods of the invention may be aggregated and/or cross linked into multimeric proteins.

As noted above, the invention also provides blood progenitor cells produced by the method, where the cells are cells that express genes that are expressed by primary human hematopoeitic stem cells including SPN, HLF and THY1. In yet another aspect of the invention, the cells may go on to become myeloid progenitors, mast cell progenitors, lymphoid progenitors and erythroid progenitors that express foetal and adult hemoglobin genes including HBG2 and HBB, T cell progenitors that express CD7, IL7R, PTCRA as well as high levels of IGLL1, SRGN and CXCR4, These cells may optionally be isolated.

In some other aspects of the invention, progenitor T cells that progress through a highly proliferative stage followed by a non-cycling stage and T cell progenitors that express high levels of class-I HLA genes including HLA-A and HLA-B and B2M are also produced. These cells can also be isolated.

In some embodiments, the timelines for cell culture for each step can be found in Table 1. In some embodiments, for step (a), it promotes blood precursor differentiation, PSCs are aggregated on day 0 and the aggregates are harvested and dissociated on day 8. In some embodiments, the aggregates are harvested and dissociated on day 9, 10, 11 or 12. For step (b), which is the endothelial hematopoietic transition phase, in preferred embodiments the cells are cultured for 5 to 7 days. In other embodiments, the cells are cultured for 2, 3 or 4 days. In other embodiments, step (b) is timed to end upon the appearance of myeloid cells in culture. Step (c) promotes T cell differentiation. In some aspects, the cells are cultured for 7 to 14 days or in some aspects of the invention longer, depending on what stage of T cell development is desired.

In some aspects, the blood progenitor cells produced or obtainable from step (b) have a transcriptional profile similar to hematopoietic stem and progenitor cells and express HLF, THY1, SPN, ERG, HOXA9, HOXA10, LCOR, RUNX1 and SPI1. In some embodiments, the cells produced can produce erythroid progenitors, megakaryocyte progenitors, mast cell progenitors, myeloid progenitors and lymphoid progenitors. These cells are likely useful for therapy or other uses in their own right. When step (b), the EHT phase is omitted, the cells produced do not have a transcriptional profile similar to hematopoietic stem and progenitor cells.

In some aspects, the process of the invention produces proT cells and later stage T cells with unique transcriptional profiles and other properties.

In some aspects the invention provides methods and/or uses of the cells produced by the methods of the present invention. For instance the blood progenitor cells themselves can be used as a source of cells in various therapies and treatments, such as in or in replacement of bone marrow transplants and be administered to a patient in need thereof. In some aspects, depending on the stage of cell development, further differentiation of the cells may occur in vivo. In other aspects the T cells (or T cell progenitors) generated by the methods of the invention may be used in immunotherapy, such as selected from the following therapies: CAR-T, engineered TCR T cell, T-regulatory cell, genetic modification therapy and other uses. As such the methods of the invention may be used to produce cells (or to source cells) that can be used in various medical treatments for a number of medical conditions. As such, the cells can, in some aspects be used to make medicaments for the use in said treatments. In some aspects the cells are in compositions, such as a pharmaceutical composition comprising the cells and a pharmaceutically acceptable carrier and/or media and/or other excipients. In some other aspects, said cells and/or compositions can be adminstered to a patient in need thereof.

These and other aspects of the disclosure are described in further detail below.

### DESCRIPTION OF DRAWINGS

FIG. 1 illustrates a three-step approach for feeder-free T cell differentiation from pluripotent stem cells: a) Schematic overview of two T cell differentiation strategies. The approach of the present invention comprises a three-step process where pluripotent stem cells are aggregated and directed to become hemogenic endothelial cells and then cultured on a cell culture substrate functionalised with recombinant DLL4 and VCAM1 in a media designed to support endothelial to hematopoietic transition. Next, the cells are grown in media optimised for T cell differentiation on a cell culture substrate functionalised with recombinant DLL4 and VCAM1. b) Representative flow cytometry analysis of iPS11 cells subjected to either a 2-step or 3-step differentiation protocol after 22 days. Gated cells express CD5 and CD7, indicative of T cell progenitors. c) Representative flow cytometry analysis of iPS11 cells subjected to either a 2-step or 3-step differentiation protocol after 29 days. Gated cells express CD5 and CD7, indicative of T cell progenitors. d) Representative flow cytometry analysis of iPS11 cells subjected to either a 2-step or 3-step differentiation protocol after 29 days. Gated cells are immature CD4 single positive T cell progenitors.

FIG. 2 illustrates a three step approach improves T cell differentiation from pluripotent stem cells: a) Yield of CD5+, CD7+ T cell progenitors produced after 22 days using either a 2-step or 3-step differentiation process. Error bars reflect standard deviation of 3 independent replicates. b.) Yield of CD5+, CD7+ T cell progenitors produced after 29 days using either a 2-step or 3-step differentiation process. Error bars reflect standard deviation of 3 independent replicates. c.) Yield of CD4ISP T cell progenitors produced after 29 days using either a 2-step or 3-step differentiation process. Error bars reflect standard deviation of 3 independent replicates.

FIG. 3 illustrated a three step approach generates T cell progenitors with a distinct transcriptional profile: a) Single cell RNA sequencing analysis performed on T cell progenitors generated from iPS11 in vitro. Unsupervised leiden clustering reveals distinct gene expression profiles for cells produced using the 3-step protocol compared to cells produced using the 2-step protocol. b) Top 20 overexpressed genes in 3-step protocol (left) and 2-step protocol (right) plotted by Z-score (wilcoxon rank-sum test). c, d and e) Violin plots comparing expression levels between protocols for house-keeping genes (c) and a selection of genes that are overexpressed in the 3-step (d) and 2-step (e) protocols.

FIG. 4 illustrated the three-step protocol produces progenitors capable of maturing into unmodified, and TCR engineered T cells: a) Flow cytometry analysis confirms that pro T cells generated from iPS11 using the 3-step differentiation protocol can continue to mature into CD8+, αβTCR+, CD3+ T cells using defined differentiation conditions. b) Flow cytometry analysis of effector and memory phenotypes of CD4+, CD8+ double positive (DP) cells and CD8 single positive (SP) T cells generated by stimulating DPs with αCD3, αCD2, αCD28 beads. Results are quantified in (c) a bar graph and (d) flow cytometry analysis of T cells engineered with a lentiviral vector encoding an αEBV T cell receptor and an mStrawberry reporter are able to mature into DP (double positive CD4+ and CD8+) and SP (single positive, CD4+ or CD8+) T cells.

FIG. 5 illustrated production of T-competent blood progenitors is Notch dependent and is enhanced by the combination of DL4 and VCAM1: a) Experimental schematic. Cells were cultured in media designed to support endothelial to hematopoietic transition on uncoated culture plates, plates coated with Fc-DLL4, with Fc-VCAM1 or both proteins for 7 days. All conditions were subsequently passaged on to plates coated with both Fc-DLL4 and Fc-VCAM1 proteins in the presence of media that supports pro T cell differentiation. b) Representative flow cytometry analysis of CD7+ and CD5+, CD7+ T cell progenitors generated in each condition at day 14. c) Quantification of the frequency and yield of T cell progenitors from (b) across conditions (n=6 technical replicates)

FIG. 6 illustrates hematopoietic stem and progenitor cells produced in the presence of DL4 and VCAM1 have a definitive transcriptome and multilineage potential: a) Universal Manifold Approximation and Projection (UMAP) and unsupervised leiden clustering analysis of single cell RNA sequencing (scRNA-seq) data obtained over the course of the described differentiation process. Ery/MK=erythroid megakaryocyte progenitors. Endothelial and hematopoietic cell types were identified on the basis of marker gene expression shown in (b) Notably, the generated cells display both lymphoid and myeloid potential and produce erythroid progenitors that express definitive globin genes including HBB.

FIG. 7 illustrates gene expression changes over time during endothelial to hematopoietic transition and progenitor T cell differentiation. New endothelial to hematopoietic cultures were initiated from cryopreserved CD34+ cells on DLL4 and VCAM1 every day for 14 days. After 7 days in media designed to support endothelial to hematopoietic transition, cells were passaged into T cell differentiation media in the presence of DLL4 and VCAM1. After 14 days, cells from each time point were collected and subjected to single-cell RNA sequencing. Expression of genes associated with endothelial, hematopoietic and T cell identity are plotted over time. Time point 1 is the initial CD34+ population and time point 15 are cells that have gone through 7 days of endothelial to hematopoietic culture and 7 days of T cell differentiation.

FIG. 8: is a schematic overview of key developmental stages during differentiation from pluripotent stem cells to T cells in accordance with the present invention.

FIG. 9: illustrates addition of a culture stage that supports endothelial to hematopoietic transition improves progenitor T cell output: a) Schematic overview of developmental stages between pluripotent stem cells and T cell progenitors and their associated immunophenotype b) Schematic overview and of a 2-step differentiation protocol. c) Representative flow cytometry analysis of the cells generated during the aggrewell blood induction (phase 1) portion of the protocol. d) Representative flow cytometry of the cells generated after enriching for CD34+ cells following aggrewell blood induction and subsequently culturing the enriched cells for 14 days in T cell differentiation media (2 step protocol). e) Schematic overview and of a 3-step differentiation protocol. f.) Representative flow cytometry of the cells generated after enriching for CD34+ cells after aggrewell blood induction and subsequently cultured for 7 days in media formulated to support endothelial to hematopoietic transition in the presence of DLL4 and VCAM1 and then for 7 additional days in T cell differentiation media (3 step protocol).

FIG. 10 illustrates quantifying T cell progenitor output across cell seeding densities: a) Schematic overview of developmental stages between pluripotent stem cells and T cell progenitors b) Schematic overview and of a 2-step and 3-step differentiation protocol. c) Quantification of the yield of CD5+, CD7+ T cell progenitors between protocols and across seeding densities as determined by flow cytometry.

FIG. 11 illustrates T cell progenitor differentiation requires DLL4 during endothelial to hematopoietic transition and is enhanced by VCAM1: a) Schematic overview of developmental stages between pluripotent stem cells and T cell progenitors b) Schematic overview of the experimental design used to assess the effect of immobilized DLL4 and VCAM1 during the endothelial to hematopoietic transition phase. c) Quantification of the yield of CD5+, CD7+ T cell progenitors after undergoing endothelial to hematopoietic transition in the presence of the indicated immobilized proteins, or an uncoated control surface.

FIG. 12 illustrates blood progenitors generated in the presence of DLL4 and VCAM1 can mature into T cells: a) Schematic overview of 3-step differentiation process b) Representative flow cytometry demonstrating blood progenitors generated in media that supports endothelial to hematopoietic transition in the presence of DLL4 and VCAM1 can progressively mature into early T cell progenitors, late T cell progenitors and mature T cells.

FIG. 13 illustrates the presence of DLL4 and VCAM1 during the endothelial to hematopoietic transition supports development of HSPC with robust T cell potential. a.) Schematic overview of chemically defined platform for producing multipotent hematopoietic progenitors and T cell progenitors from pluripotent stem cells. b.) Flow cytometry analysis of progenitor T cell output after transitioning cells from 7 days in each EHT coating condition into a common defined thymic niche for an additional 7 days. c.) Quantification of the frequency and yield of CD7+ lymphoid progenitors and CD7+, CD5+ T cell progenitors after 7 days in the thymic niche (mean+/−s.d., n=6). d.) Experimental design to assess the effect of adding or omitting the EHT culture phase prior to transferring cells into the thymic niche. e.) Immunophenotype of cells generated with or without the EHT culture stage. Numerals in (e) correspond to the schematic in (d). Plots are representative of n=3 replicates and summary statistics are shown in supplementary figure X.

FIG. 14 illustrates that EHT drastically improves progenitor T cell differentiation from PSC derived CD34+ cells. a. shows morphological transition from adherent, endothelial like cells, to non-adherent spherical cells cell cultured at days 1, 5 and 7. b.) The yield of non-adherent hematopoietic cells was quantified after 5 days in EHT under the coating conditions indicated. c.) Flow cytometry quantification of T cell progenitor frequency and yield for cells were cultured with, or without (6F) the EHT culture phase in accordance with the schematic in FIG. 1d. Analysis was performed after 21 days of culture post-CD34+ enrichment.

FIG. 15 illustrates that engineered notch signalling during EHT reduces neutrophil differentiation and promotes definitive hematopoiesis. a.) Schematic overview of the experimental design used to test how the presence of DLL4 and VCAM1 during EHT impacts the resulting HSPC. b.) UMAP projection of cells identified in scRNA-sequencing after quality control filtration. Cells are coloured by unsupervised Leiden clusters annotated by expression of known marker genes. c.) UMAPs as in (b), coloured by coating condition. d.) Expression of hematopoietic and endothelial genes by cluster. Expression is scaled across all cells to a mean of 0 and unit variance. e.) Proportion of each hematopoietic progenitor subtype identified in scRNA-seq data, quantified for each coating condition. f.) Flow cytometry quantification of neutrophil output from HSPC generated in each EHT coating condition following extended 14-day liquid in myeloid supportive media. g.) Comparison by coating condition of expression of hemoglobin genes and megakaryocyte-associated transcription factors within the MK/erythroid progenitor cluster. Expression is scaled across all cells to a mean of 0 and unit variance. h.) CFU-GEMM quantification from HSPC generated in each EHT coating condition following 14-day culture in semi-solid media (mean+/−s.d., n=6). Representative colony image shown is inset.

FIG. 16 illustrates that engineered notch signalling during EHT alters cell-cell interaction programs. a.) Inter-cellular interactions predicted by CellPhoneDB from scRNA sequencing of hematopoietic progenitors produced in each coating condition. Interacting cell types and ligand-receptor pairs are indicated for the top 5 predicted interactions in each condition. b.) Expression of the ligands and receptors involved in the most likely inter-cellular interactions plotted by coating condition. Expression is scaled across all cells within the cluster to a mean of 0 and unit variance.

FIG. 17 illustrates that VCAM1 promotes an inflammatory program and cooperates with DLL4 to enhance notch signalling and hematopoietic gene expression in HSC/MPP. a.) Expression of known notch target genes were analysed in cells within the HSC/MPP cluster from each coating condition. Expression of each gene scaled to a mean of 0 and unit variance within all HSC/MPP. b.) The geometric means of the notch target genes shown in (a) were combined on a per-cell basis to create a single-cell notch activity score. c.) Expression of genes that are confidently impacted by VCAM1 within the HSC/MPP cluster. Depicted genes were differentially expressed, both between VCAM1 vs uncoated and between DLL4+VCAM1 vs DLL4. d.) Differential pathway activity within HSC/MPP across coating conditions. Pairwise comparisons were performed between each coating condition and all others and the top 8 differentially active pathways from each comparison are depicted. Grey shaded boxes indicate comparisons where a given pathway was amongst the top 8 most differentially active. e, f, g.) Comparison of SCENIC regulon activity between coating conditions. The top 10 regulons specific to each coating condition are shown. Regulon specificity is calculated as the Jensen-Shannon distance between conditions. h.) Model of the impact of coating conditions on notch signalling and associated notch dependence of transcriptional programs and cell fate decisions.

FIG. 18 illustrates an unbiased exploration of the impact of DLL4 and VCAM1 during EHT on gene expression in PSC derived HSC/MPP. a.) Differential gene expression within HSC/MPP across coating conditions. Pairwise comparisons were performed between each coating condition and all others and the top 8 differentially expressed genes from each comparison are depicted. Grey shaded boxes indicate comparisons where a given gene was amongst the top 8 most differentially expressed.

FIG. 19 illustrates that Modelling cytokine dose responses and optimization throughout T-cell development. (a) A 6-factor orthogonal central composite design (CCD) experiment was performed at two stages of T-cell differentiation (T8+7-14 and T8+14-28). A polynomial equation fit using least-squares regression was used to model the dose response for each population of interest. (b) Predicted dose response for each population measured and for each cytokine. (c) Significant two-factor interactions between cytokines during day 7-14. (d) Significant two-factor interactions between cytokines during day 14-28. (e) In order to optimize cytokine concentrations to generate CD8+ T-cells, an ancestor-progeny relationship was assumed, where increasing the number of early phenotypes (ie. proT-cells and CD4ISP) would lead to larger numbers of DP and CD8SP T-cells later. (f) For each population of interest i, a desirability function d(Yᵢ) was defined which scales the output of each polynomial model Yᵢ(X) between [0,1]. Cytokine concentrations X that increased Yᵢ result in a desirability closer to 1 (more desirable) whereas those that decrease Yᵢ are closer to 0 (less desirable). (g) The desirability function for each population was combined using the geometric mean to provide an overall desirability score D which was optimized using the single objective basin-hopping algorithm. (h) Objective for each population of interest. Day 7-14 focused on early phenotypes while day 14-28 focused on more mature phenotypes once they were present in culture. (i) Predicted optimal cytokines for each stage. 25 random cytokine concentrations were used to initialize the basin-hopping algorithm and the top 5 most desirable solutions were kept. The solid line represents the mean of the top 5 solutions while the dotted line is the standard deviation. A larger standard deviation indicates that optimal solutions were less sensitive to that particular cytokine.

FIG. 20 illustrates that optimized cytokines enhance T-cell development. (a) Predicted optima for PSC-derived T-cell differentiation compared with previously identified optima for UCB-derived HSPC differentiation to the T-lineage. (b) To validate predictions, CD34+ HE were cultured in EHT conditions for 7 days then transferred to cytokines optimized for PSC- or UCB-derived cells. Additionally, CD34+ HE were placed directly into PSC optima without the EHT step. (c) Total cell yield for each test condition . . . (d-e) By day T8+14, PSC optima with EHT induced a higher frequency and yield of CD7+CD5+ proT-cells than without EHT or using UCB optima. (f-g) By T8+28, the frequency of CD4+CD8b+ T-cells was higher in PSC optima without EHT than with, although the yield remained higher with EHT. (h-i) After stimulation with anti-CD2/3/28+IL-21, similar frequencies of CD4-CD8b+ T-cells were present with or without EHT, but the yield was greater with EHT. Additionally, a greater number of these cells expressed a TCRab, and more expressed the CD8ab heterodimer and CD62L, suggesting these cells are developmentally similar to thymocytes preparing for thymic egress. (j) T-cells generated using PSC optima secreted IL-2 and IFNy when stimulated with PMA and ionomycin.

FIG. 21 illustrates the frequency and yield of CD7+ cells. PSC optima without EHT had the highest frequency and yield of CD7+ lymphocytes on day T8+14.

FIG. 22 is a graph showing the results of limiting dilution analysis to assess the frequency of cells with CD7+ lymphoid potential within the CD34+ population using a three-step approach (+EHT) or using a two-step approach (−EHT) as described in Example 9.

FIGS. 23 a,b, c, and d are bar graphs illustrating high-throughput sequencing of TCR beta chains from PSC-derived T cell differentiation cultures (n=3 independent differentiation wells). FIG. 23a illustrates TCRB V segment usage plotted for T cells/T cell progenitors from different sources (n=3 independent differentiation wells). PSC=pluripotent stem cell, UCB=umbilical cord blood, peripheral=primary peripheral blood derived T cells, thymus=thymocytes. Data for umbilical cord blood (UCB), peripheral blood-derived T cells and primary thymus were generated and first presented in Edgar et al, 2022 and shown again here for comparison to PSC-derived cells. Error bars=+/−s.d. FIG. 23b TCR J usage for the same populations shown in FIG. 23a. FIG. 23c CDR3 length distributions for the same populations shown in FIG. 23a. FIG. 23d Average CDR3 lengths are plotted for the cells from each cell source.

FIG. 24 a.) is scRNA sequencing data from PSC-derived HSPC integrated with a recently published dataset from primary human hematopoietic development (Calvanese et al., Nature, 2022). A full description can be found in Example 11. b.) Dotplot shows the scores for each primary cell type label (columns) broken down by the coating condition used during EHT to generate our PSC-derived HSPCs (rows). c.) illustrates the quantification of the frequency of each classified cell type plotted by EHT coating condition, after classifying PSC-derived cells into primary cell types from Calvanese et al. using a transcriptome-wide anchor-based integration strategy. d,e.) illustrate a comparison of the transcriptional identity of PSC-derived cells that were classified as HSCs with primary HSCs from different anatomical locations and developmental time points by comparing expression of genes from an “HSC maturation scorecard” established by Calvanese et al. (d) is an example regression comparing PSC-derived HSCs to their most-similar primary counterpart, HSCs from the 5-week AGM. The numbers after each primary cell type label are sample identifiers from Calvanese et al. Note that there are two biological replicates for the 5-week AGM (555 and 575). f.) is a dotplot showing expression of genes from the HSC maturation score card used for analysis in (d) and (e). Dashed box highlights our PSC-derived HSCs and their most similar primary counterpart, a sample from the 5-week AGM. g.) is a comparison of PSC-derived cells to primary cells from Calvanese et al, The two datasets were integrated sing the Scanpy ‘ingest’ function and plotted them in a UMAP. h.) illustrates the use Automated Cell Type Identification using Neural Networks (ACTINN, Ma and Pellegrini, 2020) to classify cells into their most similar primary counterparts and is further explained in Example 11.

FIG. 25 Transcribed lineage barcodes were used to track the output of individual PSC-derived hematopoietic cells and downstream lineage output were scored by single cell RNA sequencing and unsupervised clustering.

### DETAILED DESCRIPTION OF THE INVENTION

The present invention is a new method for producing blood progenitor cells, T cells and their progenitors from human pluripotent stem cells (PSCs), including embryonic stem cells and induced PSCs. Though previous processes have been developed to produce T cells from PSCs, these processes require the use of immortalized mouse feeder cells and the use of these xenogeneic feeder cells limits the clinical utility of these existing processes. In one aspect, the method of the invention is non-xenogeneic. In another aspect, the media is a serum-free defined media.

The method of the present invention comprises the following steps: 1. PSCs aggregate (naturally, through growth or under conditions that promote or permit aggregation) into 3-dimensional multi-cellular structures, these aggregates are subjected to staged media formulations that direct the cells to become mesoderm and subsequently hemogenic endothelium. 2. Next, the aggregates are disassociated and hemogenic endothelial cells are directed to differentiate into blood progenitor cells (or hematopoietic stem/progenitor cells) using a media formulation (such as a feeder-free, or serum free non-xenogenic defined media) designed to promote endothelial to hematopoietic transition while being cultured on a surface functionalised with ligands designed to activate the Notch signaling pathway, such as Notch ligands and integrin ligands. 3. The resulting blood progenitor cells are cultured in media designed to promote lymphoid specification and T cell differentiation on ligands designed to activate the Notch signaling pathway, such as Notch ligands and integrin ligands. Step 2, the EHT phase or step, substantially enhances the efficacy of the protocol compared to a protocol where step 2 is omitted.

The invention has substantial commercial potential. Blood progenitor cells derived from the methods of the present invention can be used in various therapies, including genetic modification and immunotherapies. They can also be a source of genetically modified blood progenitor cells or T cells. T cells derived from a patient's own blood and engineered to recognize a target present on tumour cells are currently being used as potent treatments for hematological cancers. Despite their efficacy, these personalized or autologous treatments are extremely expensive and this has drastically limited their widespread adoption. PSCs have the capacity for unlimited growth and thus provide a renewable and inexpensive source of starting material for producing blood progenitor cells, progenitor T cells and T cells. Making the blood progenitors, progenitor T cells and T cells from PSCs using the clinically compatible feeder-free process of the present invention could lower the cost of T cell therapy and make them more accessible. Cell therapy, including T cell immunotherapy represents a rapidly growing, multibillion dollar market with applications not only for hematological malignancy but for treating solid tumours, immunodeficiency and autoimmunity. The cells of the present invention can also be used for genetically modified and/or used for gene therapy.

The present inventors have surprisingly found that using a three stage process, to (a) differentiate the PSCs to hemogenic endothelial cells and directing their differentiation to hematopoietic cells (blood progenitor cells) and then to progenitor T cells versus a two stage process of directing the PSCs to T cell progenitors; and (b) use of ligands to activate the Notch signaling pathway processes, such as Notch ligands and integrin ligands, during the endothelial to blood precursor/progenitor transition or differentiation process enhances blood progenitor yields and T cell yields and also late T cell progenitors and mature T cell yields over the prior art which generates much lower yields and tends to primarily result in the production of early T cell progenitors. It should be noted that if one has hemogenic endothelial cells then one can start the method of the present invention in the EHT stage or process.

Further, it was not previously known that going through the hemogenic endothelial to hematopoietic cell would enhance both the production of progenitor T cells, but also differentiation to later stage progenitor T cells and mature T cells. Nor was it known that notch signaling during said hemogenic endothelial to hematopoietic cell transition stage could enhance generation of lymphoid precursors (i.e. T cell precursors and progenitors) and also yielding more late stage and mature T cells.

Although, it is still possible to obtain hematopoietic cells without activating the Notch signaling pathway, the resulting hematopoietic cells are unable to effectively become T cell progenitors and eventually T cells. The addition of the ligands during the EHT promotion step produces a qualitatively different hematopoietic cell (which has utility in its own right) and that has the potential to develop into a T cell progenitor and mature T cell. In one aspect of the invention, the present inventors have shown that this could be achieved using immobilized or adsorbed DLL4 and VCAM1. The present inventors leveraged synergistic interactions between Notch ligand Delta-like 4 and integrin ligand, vascular cell adhesion molecule 1 (VCAM-1) to enhance not only Notch signaling and progenitor T cell differentiation rates but also to enhance the hemogenic endothelial to hematopoietic transition.

### I. Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein “process” and “method” are used interchangeably. Further “step” and “phase” are also used interchangeably, as is “progenitor T cell” and “T cell progenitors”.

As used herein the cells used are cells of any subject (as defined below), including human cells. The invention is not necessarily to human cells and other cell species that have similar cell differentiation and development can work.

As used herein, the term “blood induction” refers to differentiation to hemogenic endothelial cells and or “blood induction step” refers to a “hemogenic endothelial cell generation step”.

As used herein, a “defined culture medium” refers to a chemically-defined formulation comprised solely of chemically-defined constituents. A defined medium may include constituents having known chemical compositions. Medium constituents may be synthetic and/or derived from known non-synthetic sources. For example, a defined medium may include one or more growth factors secreted from known tissues or cells. However, the defined medium will not include the conditioned medium from a culture of such cells. A defined medium may include specific, known serum components isolated from an animal, including human serum components, but the defined medium will not include serum. Any serum components provided in the defined medium such as, for example, bovine serum albumin (BSA), are preferably substantially homogeneous.

As used herein, “Delta-like-4”, “DL4”, “DLL4” and “Notch ligand DL4” refer to a protein that in humans is encoded by the DLL4 gene. DL4 is a member of the Notch signaling pathway and is also referred to in the art as “Delta like ligand 4” and “DLL4”. Herein, reference to DL4 is not limited to the entire DL4 protein, but includes at least the signaling peptide portion of DL4. For example, a commercially available product (Sino Biologicals) comprising the extracellular domain (Met 1-Pro 524) of human DLL4 (full-length DLL4 accession number NP_061947.1; SEQ ID NO: 1) fused to the Fc region of human IgG 1 at the C-terminus is a DL4 protein suitable for use herein.

“Integrin(s)” as used herein refer to a superfamily of cell adhesion receptors that bind to extracellular matrix ligands, cell-surface ligands, and soluble ligands. They are transmembrane αβ heterodimers and at least 18 α and eight β subunits are known in humans, generating 24 heterodimers. On ligand binding, integrins transduce signals into the cell interior; they can also receive intracellular signals that regulate their ligand-binding affinity.

As used herein, “Vascular cell adhesion molecule 1” and “VCAM-1” and “VCAM1” refer to a protein that in humans is encoded by the VCAM1 gene. VCAM-1 is a cell surface sialoglycoprotein, a type I membrane protein that is a member of the Ig superfamily. VCAM-1 is also referred to in the art as “vascular cell adhesion protein 1 and cluster of differentiation 106 (CD106). Herein, reference to VCAM-1 is not limited to the entire VCAM-1 protein, but includes at least the signaling peptide portion of VCAM-1 (QIDSPL (SEQ ID NO: 2) or TQIDSPLN (SEQ ID NO: 3)). For example, a commercially available mouse VCAM-1 Fc chimeric protein (R&D) that comprises (Phe25-Glu698) region of mouse VCAM-1 (full-length murine VCAM-1 accession number CAA47989; SEQ ID NO: 4) fused with the Fc region of human IgG 1 is a VCAM-1 protein suitable for use herein. Use of at least a portion of human VCAM-1 (full-length human VCAM-1 accession number P19320, NP001069, EAW72950; SEQ ID NO: 5) may also be suitable for use in the method provided herein.

As used herein “feeder-free process” is a process that is free from cells of other sources, for instance in reference to the culturing human pluripotent stem cells and derivatives and not putting in cells to the media from other sources other than hematopoietic stem cells, or “serum-free process” is a process designed to grow a specific cell type or perform a specific application in the absence of serum and is non-xenogeneic. “Non-xenogeneic” refers to not using cells or tissues from other species.

As used herein, “feeder-free medium” or “serum-free medium” refers to a cell culture medium that is a defined media and lacks animal serum and lacks undefined components. Serum-free medium may include specific, known defined serum components isolated from an animal (including human animals), such as, for example, BSA.

As used herein, a “stem cell(s)” is an undifferentiated cell that can divide to produce some offspring cells that continue as stem cells and some cells that are destined to differentiate (become specialized). They can differentiate into more specialized cells but also have the capacity for self-renewal. Stem cells are an ongoing source of the differentiated cells that make up the tissues and organs of animals and plants. Stem cells include pluripotent stem cells (PSCs), such as embryonic stem cells (ESCs) and induced pluripotent stem cells (iPSCs), and multipotent stem cells, such as cord blood stem cells, and adult stem cells, which are found in various tissues.

As used herein a “precursor cell or cells” are an intermediate cell before they become differentiated after being a stem cell. Usually, a precursor cell is a stem cell with the capacity to differentiate into only one cell type.

As used herein a “progenitor cell or cells” descend from stem cells that then further differentiate into specialized cell types (one or more types of cells). They are more specific than a stem cell and can be pushed to differentiate into its “target” cell. There are many types of progenitor cells throughout the human body. Each progenitor cell generally is only capable of differentiating into cells that belong to the same tissue or organ and typically do not have the ability for self-renewal.

The main difference between progenitor and precursor cells is that progenitor cells are mainly multipotent cells that can differentiate into many types of cells, whereas precursor cells are unipotent cells that can only differentiate into a particular type of cells.

As used herein “pluripotent stem cells (PSC)” are cells that can self-renew. Self-renewal is the capacity of the stem cells to divide indefinitely, producing unaltered cell daughters maintaining the same properties of the progenitor cell. In particular conditions or under specific signals, a stem cell is able to exit from self-renewal and engage a program leading to differentiate into specialized cell types deriving from the three germ layers (ectoderm, endoderm, and mesoderm). In general, there are two types of PSCs, embryonic stem cells (ESCs) and induced pluripotent stem cells (iPSCs). ESCs are derived from the inner cell mass (ICM) of preimplantation embryos and can be indefinitely maintained and expanded in the pluripotent state in vitro. Pluripotent stem cells can also be obtained by inducing dedifferentiation of adult somatic cells through a in vitro technology, known as cell reprogramming. Similarly, to ESC, iPSC can be expanded indefinitely and they are capable to differentiate in all the derivatives of the three germ layers.

As used herein a hemogenic endothelial cell is a specialized subset of developing vascular endothelium that acquires hematopoietic potential and can give rise to multilineage hematopoietic stem and progenitor cells. In the present invention the presence of hemogenic endothelial cells was identified by the following cell surface marker profile: CD34+ and lack of CD43(−). “Hemogenic endothelium cells” as used herein has the same meaning and are used interchangeably.

As used herein a “blood progenitor” is a hematopoietic cell that has properties of either a hematopoietic stem cell or a hematopoietic progenitor cell. It can be identified by simultaneous expression of the cell surface markers CD34 and CD43. It is capable of differentiating into cells belonging to multiple hematopoietic lineages including, but not limited to, myeloid cells, erythroid cells, megakaryocytes, lymphoid cells, mast cells, basophils and eosinophils. It has the ability to differentiate into cells from at least two of these lineages. It may, or may not be capable of self-renewal. It may be used interchangeably with the term hematopoietic stem/progenitor cell or hematopoietic stem and progenitor cells (HSPC).

“Definitive blood progenitor cells” as used herein are cells that can give rise to all the mature cells of the blood forming system. Definitive blood progenitor cells can also be defined by their molecular signature for instance as described in Calvanese, V., Capellera-Garcia, S., Ma, F. et al. Mapping human haematopoietic stem cells from hemogenic endothelium to birth. Nature 604, 534-540 (2022). https://doi.org/10.1038/s41586-022-04571-x.

As used herein hematopoietic stem cell (HSC), hematopoietic progenitor cells (HPCs) and mixed population of same (HSPCs) have the definition as noted above. In the present invention the presence of HSPCs were identified by the following cell surface marker profile CD34+ and CD 43+.

As used herein, the terms “progenitor T cell” and “pro-T cell” and “T cell progenitor” refer to a cell that is derived (directly or indirectly) from a pluripotent stem cell or a CD34+ hematopoietic stem and/or progenitor cell and expresses CD7+ (human system) or CD25+CD90+ (mouse system), and has the capacity to differentiate into one or more types of mature T cells. A mature T cell includes cells that express a combination of CD4, CD8 and CD3 cell surface markers.

As used herein an early T cell progenitor is a progenitor cell that is committed to generating T cells but can produce multilineages of T cells. They are cells that in the stage of differentiation are between the multipotent hemopoietic stem cell (HSC) and the fully committed precursors undergoing T cell receptor (TCR) gene rearrangement. In the present invention, the presence of early T cell progenitors was identified by the following cell surface marker profile CD7+ and CD 5+.

As used herein a late T cell progenitor is a progenitor cell differentiated (directly or indirectly) from early T cell progenitor cell that are more committed to generating mature T cells. In the present invention, the presence of late T cell progenitors was identified by the following cell surface marker double positive (DP) thymocyte profile CD4+ and CD 8+ or single positive (CD 8+).

As used herein a mature T cell is a T cell that has developed its own T cell receptor (TCR+), or expresses an engineered TCR on the cell surface, or is a CD8+ single positive T cell that expresses a chimeric antigen receptor (CAR) or a CAR expressing T cell engineered to lack TCR. In the present invention, the presence of mature T cells was identified by the following cell surface marker profile CD3+ and CD 8+ and TCR+. Mature T cells may also be identified by a cell surface marker profile CD3+ and CD8+ and TCR+, or CD8+ CAR+.

As used herein “isolated” means non-naturally occurring cells or cell populations not themselves found in nature, for instance in a different cellular environment, culture or media that is not found in nature and that has utility in isolated form that is not present in naturally occurring non-isolated cells or cell populations. In the present invention, for instance, cells produced by the methods of the present invention, for instance the resulting or obtainable blood progenitors and progenitor T cells can, in one embodiment be isolated by cell sorting/isolation methods known in the art, and/or by their selecting for their characteristic one or more cell surface markers.

As used herein “aggregating/aggregated pluripotent stem cells into 3-dimensional multi-cellular structures” means pluripotent stem cells that naturally or are induced by culture media conditions aggregate and form 3-dimensional multi-cellular structures. The groups of multiple cells adhere to each other but do not adhere to the cells culture vessel. In some embodiments, the aggregates are more than one, more than two, more than five or generally from 10-1000 cells. See for instance Ungrin, M. D., Joshi, C., Nica, A., Bauwens, C. & Zandstra, P. W. Reproducible, ultra high-throughput formation of multicellular organization from single cell suspension-derived human embryonic stem cell aggregates. PLoS One 3, e1565, doi:10.1371/journal.pone.0001565 (2008).

As used herein “culturing pluripotent stem cells under conditions that promote differentiation to hemogenic endothelial cells” means culturing said cells under conditions (including media, temperature, density and any other culture conditions) that would promote said differentiation. Further “subjecting the pluripotent stem cells to staged media formulations that direct the cells to become mesoderm and subsequently hemogenic endothelial cells” means using staged media formulation or defined culture medium for instance as outlined in Table 1 to culture and promote differentiation of PSCs to hemogenic endothelial cells. See also for instance other potential EHT media Sugimura, R. et al. Haematopoietic stem and progenitor cells from human pluripotent stem cells. Nature 545, 432-438, doi:10.1038/nature22370 (2017). Further, media that supports production of hemogenic endothelium in aggregates similar to what is used in the present invention includes Sturgeon, C. M., Ditadi, A., Awong, G., Kennedy, M. & Keller, G. Wnt signaling controls the specification of definitive and primitive hematopoiesis from human pluripotent stem cells. Nat Biotechnol 32, 554-561, doi:10.1038/nbt.2915 (2014).

Alternate media systems that can support hemogenic endothelium induction include: Ng, E. S. et al. Differentiation of human embryonic stem cells to HOXA(+) hemogenic vasculature that resembles the aorta-gonad-mesonephros. Nat Biotechnol 34, 1168-1179, doi:10.1038/nbt.3702 (2016) and Uenishi, G. I. et al. NOTCH signaling specifies arterial-type definitive hemogenic endothelium from human pluripotent stem cells. Nat Commun 9, 1828, doi:10.1038/s41467-018-04134-7 (2018),

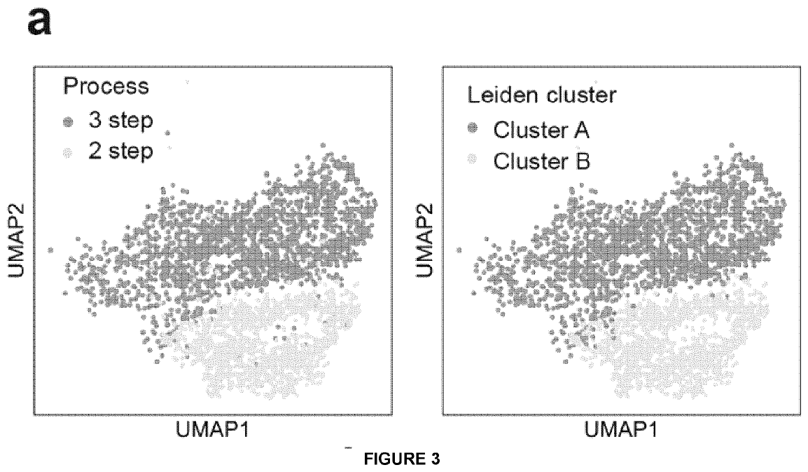

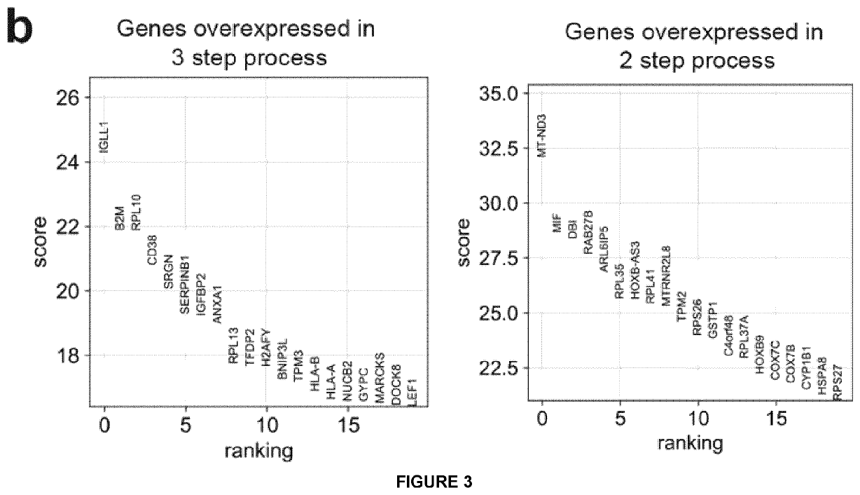

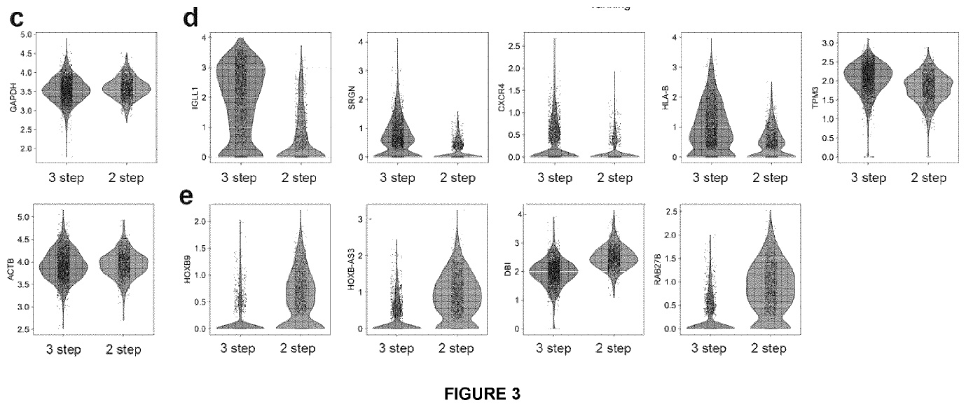

Ng et al, states “Differentiation of hESC lines was performed using the spin EB method in APEL medium65 supplemented for the first 4 d with 20 ng/ml recombinant human (rh) bone morphogenetic protein 4 (BMP4, R&D Systems), 25 ng/ml rh vascular endothelial growth factor (VEGF, PeproTech) and 25 ng/ml rh stem cell factor (SCF, PeproTech), 10 ng/nl rh ACTIVIN A (R&D Systems) and 10 ng/ml rh FGF2 (PeproTech) (FIG. 3a). Where indicated, cultures included additional CHIR99021 3 μM (Tocris Biosciences) and/or SB431542 3-4 μM (Cayman Chemicals). After 4 d, the differentiation medium on the spin EBs was changed to APEL medium supplemented with 50 ng/ml rhVEGF, 20 ng/ml BMP4, 10 ng/ml FGF2, 50 ng/ml rh SCF and 30 ng/ml rh insulin-like growth factor 2 (IGF2, PeproTech). At d7-8 of differentiation, EBs were transferred onto growth factor reduced (GFR)-Matrigel-coated, 6-well plates at 20-30 EBs/well in APEL medium including 50 ng/ml rhVEGF, 100 ng/ml rh SCF, 50 ng/ml rh interleukin (IL)-3 (PeproTech), 25 ng/ml rh IL-6 (PeproTech), 25 ng/ml rh thrombopoietin (TPO, Peprotech), 25 ng/ml rh FLT3 receptor ligand (FLT3L, PeproTech), 3 U/ml rh erythropoietin (EPO, PeproTech), 10 ng/ml FGF2, 50 ng/ml rh SCF and 20 ng/ml rh insulin-like growth factor 2 (IGF2, PeproTech).”

Uenishi, G. I. et al., states, “On Day −1, hPSCs were singularized and plated on collagen IV-coated plates (0.5 μg/cm²) at a cell density of 7500 cells/cm² in E8 media supplemented with 10 uM Rock inhibitor (Y-27632, Cayman Chemicals). On Day 0, the media was changed to IF9S media supplemented with BMP4, FGF2 (50 ng ml⁻¹), Activin A (15 ng ml⁻¹, Peprotech), LiCl (2 mM, Sigma), and ROCK inhibitor (0.5 μM, Cayman Chemicals) and cultured in hypoxia (5% O₂, 5% CO₂). On day 2, the media was changed to IF9S media supplemented with FGF2, VEGF (50 ng ml⁻¹, Peprotech), and 2.5 μM TGFβ inhibitor (SB-431542, Cayman Chemicals).” Cells harvested on day 4”.

As used herein “dissociating the aggregated cells” or “dissociating the aggregated PSCs, mesoderm and/or hemogenic endothelial cells or mixtures thereof” means using mechanical or enzymatic methods to separate multicellular aggregates into single cells, for instance using trypsin, collagenase or TrypLE Express.

The method of the invention optionally comprises enrichment of CD34+ cells. As used herein, “enriching the CD34+ population which contains within it hemogenic endothelial cells, and directing the hemogenic endothelial cells to differentiate into hematopoietic cells (blood progenitor cells) using a media formulation designed to promote endothelial to hematopoietic transition” includes, but is not necessarily limited to binding CD34+ cells with antibodies conjugated to fluorescent molecules, or conjugated to molecules that can be bound by magnetic or paramagnetic beads and subsequently selecting for CD34+ cells, for instance by fluorescence activated cells sorting or binding the cells to a substrate, and culturing the CD34+ enriched cells in the media described in Table 2.

As used herein, “culturing the hematopoietic cells in media designed to promote lymphoid specification (lymphopoiesis), differentiation into progenitor T cells and T cell differentiation” means growth media that contains factors sensed by hematopoeitic cell that lead to their differentiation into progenitor T cells, for instance in some embodiments consisting of a common basal media such as IMDM, RPMI, aMEM, SFEMII or StemPro34 and supplemented with IL-7, Flt3L and one or more of the additional factors SCF, TPO, CXCL12, TNFa, IL3 or other additional small molecules such as apoptosis inhibitors, metabolites, vitamins, bovine serum albumin, human serum albumin, or additional components.

As used herein “a surface functionalised with ligands designed to activate the Notch signalling pathway” means a surface that can be any surface that can be coated or where ligands can be adsorbed and/or immobilized which can come into contact with the cells in the culture. Such surface can include but is not necessarily limited to: a tissue culture vessel such as a tissue culture plate, flask or bioreactor, beads or hydrogels. Manufactured or human made surface or other suitable surfaces.

Further “ligands that can activate and/or enhance activation of the Notch signaling pathway” include but are not limited to Notch ligands DLL1, DLL3, DLL4, JAG1 and JAG2. In a preferred embodiment of the invention said ligands are selected from Notch ligands, such as DLL4, and integrin ligands, such as VCAM1. In some embodiments of the invention the integrin ligands are integrin α4β1 ligands. In other embodiments, the integrin ligand is VCAM1. The Notch ligands, such as DLL4 interacts with, and activates Notch receptors and the integrin ligands, which in some embodiments is VCAM1 is a cell adhesion molecule that binds to the integrin α4β1, promotes interaction between the cell and the functionalised surface, enabling additional interaction between the cell and the Notch ligand, such as DLL4 and together enhance activation of the Notch signaling pathway when they come into contact with the cell surface. The present inventors have shown synergistic effects of immobilized or adsorbed DLL4 and VCAM1 ligands in the production of HPSCs and blood progenitor cells and also in the production of progenitor T cells and more mature progenitor T cells and mature T cells. This is distinct and an improvement over use of such immobilized ligands in just the HPSC or blood progenitor to T cell progenitor stage of the process.

As used herein xenogeneic refers to denoting, relating to, or involving tissues or cells belonging to individuals of different species. While non-xenogeneic has the converse meaning.

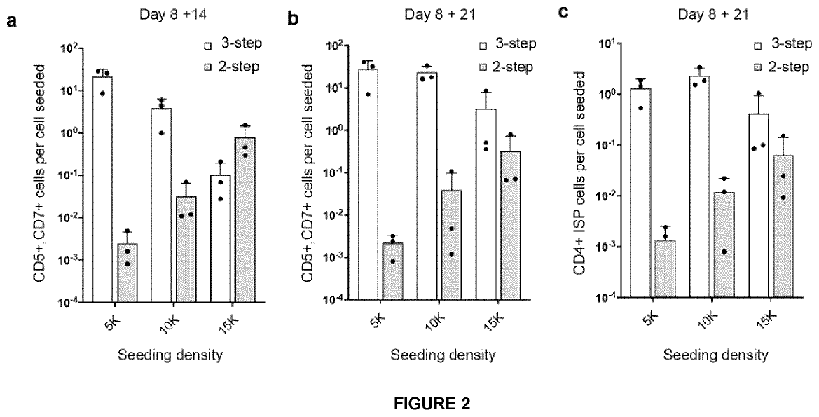

As used herein, “Multi linage development capacity” means a progenitor cells that can give rise to 3 or more district blood cell lineages. Example of lineages from blood progenitor cells includes but is not necessarily limited to red blood cells, T cells, B cells, macrophages, erythrocytes, megakaryocytes, granulocytes, neutrophils, natural killer (NK) cells, mast cells, eosinophils (e.g. Dzierzak, E. et al, Blood Development: Hematopoietic Stem Cell Dependence and Independence, Cell Stem Cell, vol. 22, Issue 5, pp 639-651, (May 3, 2018), FIG. 2).

When cells are cultured in media or staged media, the invention is not limited to the media and our stages described herein in Tables 1-4. A component could be substituted with another component or components of similar function and purpose within the media could be used in addition to or in replacement of the specific components listed and amounts and timing of stages can vary accordingly. Further “base media” as used herein could be any base media that can be any suitable media designed and optimized for growing and/or culturing the particular cell type or known to support the particular cell growth or cells.

As used herein the terms, “administration,” “administering” and variants thereof refers to introducing a composition or agent (e.g., nucleic acids, in particular cDNA) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the term “exogenous” is meant to refer to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, as used herein, the term “endogenous” refers to a substance that is native to the biological system or cell.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. In some preferred aspects of the invention, the methods of producing the blood progenitor cells and cells derived therefore, such as the T cell progenitors in the invention are done ex vivo.

The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “subject” as used herein refers to a human or animal, to whom treatment, including prophylactic treatment, with the ceDNA vector according to the present invention, is provided. Usually the animal is a vertebrate such as, but not limited to a primate, rodent, domestic animal or game animal. Primates include but are not limited to, chimpanzees, cynomolgous monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the phrase an “effective amount” is an amount sufficient to produce the desired effect, e.g., enhance cell culture, differentiation into various cell types, inhibition of expression of a target sequence in comparison to the expression level detected in the absence of a therapeutic nucleic acid. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art.

As used herein, the terms “therapeutic amount”, “therapeutically effective amount”, an “amount effective”, or “pharmaceutically effective amount” of an active agent are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, preferred materials and methods are described herein.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### II. Process of Producing Blood (Hematopoietic) Progenitor Cells and T Cell Progenitor Cells from Pluripotent Stem Cells and/or Hemogenic Endothelial Cell Through EHT

Described herein is a method for producing blood progenitor and T cell progenitor cells and to cells produced or obtainable by the process and the use of said cells, the method including or comprising:
a. Subjecting pluripotent stem cells, optionally aggregated pluripotent stem cells that are 3-dimensional multi-cellular structures, to staged media formulations that direct the cells to become mesoderm and subsequently hemogenic endothelial cells; and
b. directing the hemogenic endothelial cells to differentiate into blood progenitor cells using a media formulation designed to promote endothelial to hematopoietic transition (EHT) while being cultured on a surface functionalised with ligands designed to activate and/or enhance the Notch signaling pathway (e.g. Notch ligands such as DLL4, and integrin ligands such as vascular cell adhesion VCAM1). In some embodiments, the process comprises dissociating the aggregated cells or culturing the cells under conditions that promote or are conducive to dissociation) and/or optionally enriching the CD34+ population which comprise within it hemogenic endothelial cells, and

In a preferred aspect of the method and process of the invention as described herein, the method(s) and process(es) are conducted under non-xenogenic conditions, for instance in a defined culture medium which is feeder-free and does not comprise non-defined serum.

In some aspects, the invention provides a method for producing blood progenitor cells comprising a blood induction step, and an endothelial to hematopoietic transition step. As used herein, the term “blood induction” refers to differentiation to hemogenic endothelial cells and or “blood induction step” refers to a “hemogenic endothelial generation step”. The resulting blood progenitor cells can then be further differentiated or committed to lymphoid specification (lymphopoiesis), and differentiation into progenitor T cells and further T cell differentiation.

In some aspects, the blood induction step comprises subjecting the pluripotent stem cells to to staged media formulations that direct the cells to commit to mesoderma and subsequently hemogenic endothelial cells wherein optionally some of the pluripotent stem cells are aggregated pluripotent stem cells. For instance, see the Examples and Table 1. However, time lines for various stages for the media, may vary and be adjusted.

Thus in some embodiments, the pluripotent stem cells are cultured under conditions that promote or are conducive to aggregation, for instance into 3-dimensional multi-cellular structures. In some embodiments, whether or not actively promoted or conducive conditions are selected for, the cell culture may comprises aggregated cells. Thus, in some aspects, the pluripotent stem cells may be aggregated naturally or through chemical induction and/or by mechanical or physical means (such as agitation, centrifugation and.or stirring). In some other aspects, the 3-dimensional multi-cellular structures are 2 or more cells, 5 or more cells, or 10-1000 cell structures.

The endothelial to hematopoietic transition step may optionally comprise dissociating aggregated cells (e.g., optionally culturing the cells under conditions that dissociate or promote dissociation of cells), and optionally enriching the CD34+ cell population which comprises hemogenic endothelial cells. In some aspects, aggregated cells can be dissociated by incubating the cells with collagenase or trypsin or TrypLE Express or dissociation by mechanical force or dissociation by culturing the aggregates on a tissue culture treated surface. In some embodiments, the CD34+ cell population enrichment may comprise binding cells with anti-CD34 antibodies conjugated to fluorescent molecules, or conjugated to molecules that can be bound by magnetic or paramagnetic beads and subsequently selecting for CD34+ cells by fluorescence activated cells sorting or magnetically capturing the cells. The invention is not necessarily limited to the specific media and time lines for media culture changes in the examples or described herein. Other conditions/media may be known to those in the art.

In some aspects, the endothelial to hematopoietic transition step comprises culturing the hemogenic endothelial cells under conditions that commit and direct the cells to differentiate into hematopoietic stem and progenitor cells, wherein the conditions comprise using a media formulation designed to promote endothelial to hematopoietic transition (EHT) while being cultured on a surface functionalised with ligands designed to activate and/or enhance the activation of the Notch signaling pathway, such as Notch ligands and integrin ligands. In some aspects, the ligands are adsorbed or immobilized on the surface. In other aspects, the Notch signaling ligand is DLL4 and the integrin ligand is VCAM1. In some aspects of the invention, the media formulation designed to promote endothelial to hematopoietic transition (EHT) comprises the media of Table 2. In some other aspects, the surface functionalised with ligands designed to activate and/or enhance the Notch signaling pathway is selected from: a two dimensional tissue culture surface; a tissue culture plate; the surface of beads; the surface of hydrogels; manufactured or human made and other suitable surfaces.

In some further aspects the method results in producing blood progenitor cells, thus one my obtain a cell culture comprising blood progenitor cells and optionally isolate and select for said cells, for instance using one or more genetic expression and/or one or more cell surface markers characteristic of said cells as further described herein. For instance, in one aspect the blood progenitor cells obtainable by the method can be isolated, tracked or identified via cell surface markers CD34+ and CD43+, or CD43+ and as noted in the examples.

As noted above, in a preferred aspect of the method of the invention, the method(s) and process(es) of the invention as described herein are conducted under non-xenogenic conditions in a defined culture medium which is feeder-free and non-defined serum free.

In a further aspect, the invention provides a method for producing T cell progenitor cells comprising culturing the blood progenitor cells produced using the method described herein in media and under culture conditions designed to promote lymphoid specification (lymphopoiesis), differentiation into progenitor T cells and T cell differentiation. In one aspect, the media and culture conditions to promote lymphoid specification and differentiation into progenitor T cells and T cell differentiation comprises culturing on a surface functionalised with ligands designed to activate T cell development. In some embodiments, ligands are designed to activate and/or enhance activation of the Notch signaling pathway, such as the use of Notch ligands and integrin ligands). In some aspects, the ligands are adsorbed or immobilized on the surface. In other aspects, the Notch signaling ligand (or Notch ligand) is DLL4 and the integrin ligand is VCAM1. In some aspects of the invention, the media formulation designed to promote T cell progenitor formation comprises the media of Table 3. In some other aspects, the surface functionalised with ligands designed to activate the Notch signaling pathway is selected from: a two dimensional tissue culture surface; a tissue culture plate; the surface of beads; the surface of hydrogels; manufactured or hand made and other suitable surfaces.

In some further aspects the method results in producing progenitor T cells, thus one my obtain a cell culture comprising progenitor T cells (e.g. early and later stage progenitor T cells) and optionally isolate and select for said cells, for instance using one or more genetic expression and/or one or more cell surface markers characteristic of said cells as further described herein. For instance, in one aspect the blood progenitor cells obtainable by the method can be isolated, tracked or identified via cell surface markers CD5+ and CD7+ or later stage cells comprising CD4+ and CD8+, or CD8+, and as noted in the examples.

In some aspects, the invention provides a method further comprising steps to differentiate the T cell progenitors to mature T cells by culturing them in media designed to support T cell development on ligands designed to activate and/or enhance the Notch signalling pathway (such as Notch ligands and integrin ligands), wherein in one embodiment the ligands are adsorbed or immobilized on or in a surface.

In some further aspects, the invention provides an isolated T cell or isolated T cell population that express αβTCR, CD3 and CD8α and CD8β produced or obtainable using the methods of the invention.

In some aspects, the use of the T cells produced by the method or the isolated cells or cell population(s) can be used for immunotherapy, such as CAR-T, engineered TCR T cells, T-regulatory cells, genetic modification therapy or other uses.

In some embodiments of the invention, the PSC population is engineered to provide the cells with additional functionality such as hypoimmunity or to add cancer targeting moieties such as engineered TCR or CAR. In other embodiments the engineered TCR or CAR are added at specific stages of the process including during the EHT step, the CD34 HSPC step, the progenitor T cell step or even directly to mature T cells.

Cells Produced by/Obtainable from the Method

The invention also relates to the cells produced or obtainable by the method/process, said cells being capable of isolation and have utility in various applications.

The blood progenitor cells produced by the invention express genes that are expressed by primary human hematopoietic stem cells including SPN, PTPRC, HLF and THY1. In another embodiment, the blood progenitor cells express HLF, THY1, SPN, ERG, HOXA9, HOXA10, LCOR, RUNX1 and SPI1, and are capable of giving rise to myeloid progenitors, mast cells, lymphoid progenitors and erythroid/megakaryocyte progenitors that express foetal and adult haemoglobin genes. The hematopoietic cells produced by the invention can differentiate to become myeloid progenitors, mast cell progenitors, lymphoid progenitors and erythroid progenitors that express foetal and adult hemoglobin genes including HBG2 and HBB. They can be isolated and have utility in many applications.

In other aspects the blood progenitor cells produced by the method in the EHT induction step are

The progenitor T cells produced by the invention express CD7, IL7R, PTCRA as well as high levels of IGLL1, SRGN and CXCR4. In another embodiment the progenitor T cells produced by the invention express CD7, BCL11B, IGLL1 and CXCR4. The T cell progenitors produced by the invention progress through a highly proliferative stage followed by a non-cycling stage. The T cell progenitors produced by the invention express high levels of class-I HLA genes including HLA-A and HLA-B and B2M. They can be isolated and thus a source of isolated progenitor T cells. They can be isolated and have utility in many applications.

### III. Uses

In some aspects the invention provides methods and/or uses of the cells produced by the methods of the present invention. For instance the blood progenitor cells themselves can be used as a source of cells in various therapies and treatments, such as in or in replacement of bone marrow transplants and be administered to a patient in need thereof. In some aspects, depending on the stage of cell development, further differentiation of the cells may occur in vivo. In other aspects the T cells (or T cell progenitors) generated by the methods of the invention may be used in immunotherapy, such as selected from the following therapies: CAR-T, engineered TCR T cell, T-regulatory cell, genetic modification therapy and other uses. As such the methods of the invention may be used to produce cells (or to source cells) that can be used in various medical treatments for a number of medical conditions. As such, the cells can, in some aspects be used to make medicaments for the use in said treatments. In some aspects the cells are in compositions, such as a pharmaceutical composition comprising the cells and a pharmaceutically acceptable carrier and/or media and/or other excipients. In some other aspects, said cells and/or compositions can be adminstered to a patient in need thereof.

### IV. Kits

The present disclosure contemplates kits for carrying out the methods provided herein. In some embodiments, samples of such kit components are included in the summary of invention above, such as including but not limited various culture media or components thereof, cells, functionalized surfaces, ligands and other components to carry out the methods and/or uses of the present invention. Such kits typically comprise two or more components required for generation of blood progenitor cells and/or pro-T cells. Components of the kit include, but are not limited to, one or more of compounds, reagents, containers, equipment and instructions for using the kit. Accordingly, the methods described herein may be performed by utilizing pre-packaged kits provided herein.

In an embodiment, a kit for use to generate blood progenitor and/or pro-T cells from PSCs or HSPCs in vitro is provided. The kit comprises DL4 and VCAM-1. In an embodiment, the DL4 is adsorbed or immobilized to a substrate. In an embodiment, the VCAM-1 is adsorbed or immobilized to a substrate.

In an embodiment, the kit further comprises media that promoted EHT, such as the components listed in Table 2. The invention also includes variations to the media and components as may be known to persons of skilled in the art.

In an embodiment, the kit further comprises a T cell progenitor differentiation medium, preferably comprising growth factors, such as SCF, Flt3L, IL7 and/or TPO, in hematopoietic amounts. For example, amounts the growth factors may be as follows: 10-50 ng/ml_ (mouse cultures) and about 100 ng/ml_ (human cultures). In some embodiments, instructions for use of the kit to generate pro-T cells from stem and/or progenitor cells, such as PSCs or HSPCs, in vitro are provided.

The instructions may comprise one or more protocols for: preparing DL4 and, optionally, preparing VCAM-1 components; providing DL4 and/or VCAM-1 components to a culture system; culture conditions, such as time, temperature, and/or gas incubation concentrations; harvesting protocols; and protocols for identifying blood progenitor (HSC, HPC, HSPC), pro-T cells and, optionally, more mature T cells.

The kit may further include materials useful for conducting the present method such as, for example, culture plates, welled plates, petri dishes and the like.

### EXAMPLES

The following examples are provided by way of illustration not limitation.

### Methods

Human Pluripotent Stem Cell (hPSC) Culture

The human pluripotent stem cell line iPS11 (Alstem Cell Advancements) was cultured on tissue culture-treated plasticware pre-coated with a basement membrane extract (Geltrex, Life Technologies A1413302) in serum-free media (mTeSR1, Stemcell technologies 85850) supplemented with Penicillin-Streptomycin (Invitrogen, 15140122-0.5% V/V). The iPS11 cell line is a footprint-free human iPS (induced pluripotent stem) cell line (Cat #iPS11) was derived from human foreskin fibroblasts (HFFs) by ectopic expression of OCT4, SOX2, KLF4, and L-MYC genes using Alstem episomal plasmids. The cells are derived using morphological selection criteria and without the use of fluorescent marker or drug selection. When cultured under standard human embryonic stem (ES) cell culture conditions, the morphology of human iPS cells are identical to that of human ES cells. The cells express the pluripotency markers OCT4, SSEA-3, Nanog, and endogenous alkaline phosphatase. High viability, low passage iPS cells have been pre-adapted to serum-free, feeder free culture conditions.

Media was replaced daily and cells were maintained at 37 C, 5% CO₂. Following thawing or passaging, media was additionally supplemented with the ROCK inhibitor Y-27632 (Sigma-Aldrich Y0503-5MG) at 7.5 uM and 5 uM concentrations respectively. To passage, cells were partially dissociated to small aggregates with 1×TrypLE Express (Thermofisher 12605028) for 1 to 4 minutes at 37 C followed by scraping.

### Aggregation and CD34+ Induction

The hPSCs were grown to ˜90% confluency and dissociated to single cells using recombinant cell-dissociation enzymes (TrypLE Express, Life Technologies 12605028) for approximately 3 minutes at 37° C. Cells were counted and resuspended in 2 mL per well T0 media and deposited into microwell plates (AggreWell 400 6-Well plates, StemCell Technologies 34425) prepared according to the manufacturer's instructions. Cells were seeded at a density of 180 cells per microwell and aggregated by centrifugation at 200×g for 5 minutes. For the duration of the CD34+ induction, cells were cultured at 37° C. in a hypoxia incubator. 24 hours after seeding, 2 mL of day 1 media was added to each well. 42 hours after initiating the differentiation, media was aspirated and replaced with 2 mL per well day 1.75 media. The following day, an additional 2 mL of day 1.75 media was added to each well. 96 hours after initiating the differentiation, the day 1.75 media was aspirated and replaced with 2 mL/well day 4 media. 48 hours later, the media in each well was supplemented with 2 mL of day 6 media. The following day, a subset of aggregates were harvested and dissociated with TryPLE Express, washed with HBSS+2% FBS, stained with antibodies against CD34, CD43, CD73 and CD184 and analysed by flow cytometry. 192 hours after initiating the differentiations, aggregates were collected and pelleted by centrifugation at 200×g for 5 minutes. Spent media was aspirated and aggregates were dissociated in 3 mL TryPLE supplemented with DNaseI for 10 to 15 minutes. Cells were pipetted to a single cell suspension, washed and counted. CD34+ cells were enriched using the CD34 positive selection kit (Miltenyi Biotec, 130-046-702) according to the manufacturer's instructions. CD34+ enriched cells were either cryopreserved using CryoStor CS10 (StemCell Technologies, 07930) or used immediately for downstream culture.

### Staged Media Formulation

PSCs were cultured under the media conditions in Table 1. On day 0 the aggregates are generated in the day 0 media and over the following days are differentiated as aggregates using the components and timings indicated in the table. The invention is not limited to the particular media or staged media noted below. For instance, the base media can be any suitable media designed and optimized for growing and/or culturing the particular cell type or known to support the particular cell growth or cells. In addition the other components could be substituted with other components of similar function, and amounts can vary accordingly

TABLE 1
Media Formulations Used to Promote Blood
Precursor Differentiation (From stage 1)

| Day | Day | Day | Day | Day | Day | Day | Day |
|---|---|---|---|---|---|---|---|
| 0 | 1 | 1.75 | 3 | 4 | 5 | 6 | 7 |

Ascorbic acid (50 ug/mL)
1-thioglycerol (0.04 ul/mL)
Transferrin (150 ug/ml)

BMP4 (10 ng/mL)
Y-27632
(5 uM)

bFGF (5 ng/ml)

SB-431542 (6 uM)
CHIR-99021 (3 uM)

VEGF(15 ng/mL)
II-6 (10 ng/mL)
IL-11 (5 ng/mL)

EPO (2 U/mL)
IGF-I (25 ng/mL)
SCF (25 ng/mL)
Base media is StemPro-34 SFM (Thermo Fisher Scientific, 10639011) Medium with 1% Glutamax ™ (L-alanyl-L-glutamine dipeptide in 0.85% NaCl)

### Endothelial-To-Hematopoietic Transition (EHT) Culture

CD34+ cells generated above were used as input for EHT culture. Coating solution was prepared using sterile PBS combined with 15 ug/mL Fc-tagged DLL4 (Cedarlane Labs 10171-H02H-100) and 2.5 ug/mL Fc-tagged VCAM1 (R&D Systems, 643-VM-050). Tissue culture-treated 96 well plates (Fisher Scientific, 12-556-008) were pre-coated with 50 uL of coating solution overnight at 4 C. Coating solution was aspirated and plates were washed with PBS immediately prior to use. CD34+ enriched cells were resuspended in EHT media (Table 2) at a concentration of 1×10⁵ cells per mL. 100 uL (10,000 cells) were seeded on to each well of the 96 well plate. Cultures were incubated at 37 C, 5% CO₂ for 5 or 7 days and non-adherent cells were harvested by gentle pipetting.

The media formulation used to promote endothelial to hematopoietic transition is show in in Table 2. This media is used after the cells are disaggregated and optional CD34+ cell enrichment. The invention is not limited to the particular media or staged media noted below. For instance, the base media can be any suitable media designed and optimized for growing and/or culturing the particular cell type or known to support the particular cell growth or cells. In addition the other components could be substituted with other components of similar function and amounts can vary accordingly. Examples of suitable concentration ranges are noted in Table 2. Further general functions of the components are provided, so other components having similar function and purpose within the media could be used in addition to or in replacement of the specific components listed.

TABLE 2
Media formulation used to promote endothelial
to hematopoietic transition

| | Concen- | | Concen- | | |
|---|---|---|---|---|---|
| | tration | | tration | | Function, other |
| Reagent | Used | | Range | | potential components |
| 1- | 3 | μl/ml | 1-5 | uL/mL | Or other components |
| thioglycerol | | | | | that activate cellular |
| [MTG] | | | | | proliferation |
| (Sigma)* | | | | | |
| Ascorbic | 50 | μg/ml | 25-100 | ug/mL | Or other components |
| Acid | | | | | that can act as an |
| (Sigma) | | | | | antioxidant, |
| | | | | | citrriline/NOS agonist |
| Transferrin | 150 | μg/ml | 50-200 | ug/mL | Or other components |
| (Roche) | | | | | that may mediate iron |
| | | | | | uptake |
| bFGF | 5 | ng/mL | 1-10 | ng/mL | Or other components |
| | | | | | that are hematopoietic |
| | | | | | growth factors |
| VEGF | 5 | ng/mL | 1-10 | ng/mL | Or other components |
| | | | | | that synergises with |
| | | | | | IL03 to shorten cell |
| | | | | | cylce |
| IL-6 (dilute | 10 | ng/mL | 10-50 | ng/mL | Or other components |
| stock 1/10) | | | | | that synergises with |
| | | | | | IL-3 to shorten cell |
| | | | | | cycle |
| IL-11 | 5 | ng/mL | 0-10 | ng/mL | Optional component |
| | | | | | and in some |
| | | | | | embodiments may be |
| | | | | | omitted. Also can use |
| | | | | | other components that |
| | | | | | stimulate HSC |
| | | | | | proliferation |
| TPO | 30 | ng/mL | 10-50 | ng/mL | Or other components |
| | | | | | that regulate platelet |
| | | | | | production |
| IGF-I | 25 | ng/mL | 15-30 | ng/mL | Or other growth |
| | | | | | hormone |
| SCF | 50 | ng/mL | 50-100 | ng/mL | Or other components |
| | | | | | that promote blood cell |
| | | | | | survival, proliferation |
| | | | | | and differentiation |
| IL-3 (dilute | 10 | ng/ml | 10-30 | ng/mL | Or other components |
| stock 1/10) | | | | | that induce |
| | | | | | proliferation and |
| | | | | | differentiation |
| ROCKi | 10 | uM | 3-10 | uM | Or other components |
| | | | | | that limit cell death |
| BMP4 | 10 | ng/ml | 5-20 | ng/mL | Or other components |
| | | | | | that support HSC |
| | | | | | development |
| FLT3L | 10 | ng/ml | 5-20 | ng/mL | Or other components |
| (dilute | | | | | that stimulate HSC |
| stock 1/10) | | | | | proliferation |

Base media is StemPro-34 SFM (Thermo Fisher Scientific, 10639011)with 1% Glutamax ™ (L-alanyl-L-glutamine dipeptide in 0.85% NaCl) or other suitable Base Media.

In addition, in some embodiments, one or more of EPO (e.g. 2 U/ml), Angiotensin II (e.g., 10 ug/ml) and Losartan Potassium (e.g. 100 uM) may be present in the media.

### Progenitor T Cell Differentiation

Non-adherent cells harvested from EHT cultures were pelleted by centrifugation at 300×g for 5 minutes. Spent media was aspirated and cells were resuspended in T cell differentiation media (Table 3) at a split at a ratio of 1:2, 1:3 or 1:4. Cells were seeded in 100 uL per well of a 96 well plate pre-coated with DLL4 and VCAM1 as described above. 3 to 4 days after seeding, cells were fed with an additional 100 uL per well of T cell differentiation media. 7 days after seeding, cells were harvested by pipetting and seeded on to freshly coated plates in T cell differentiation media or SFEM II (StemCell technologies, 09605) supplemented with 1×StemSpan T Cell Progenitor Maturation Supplement (StemCell Technologies 09930) and cultured for an additional 7 to 21 days, during which time cells were subjected to half media changes every 3 to 4 days.

Media formulation used to promote T Cell differentiation is shown in Table 3. The invention is not limited to the particular media or staged media noted herein. For instance, the base media can be any suitable media designed and optimized for growing and/or culturing the particular cell type or known to support the particular cell growth or cells. In addition the other components could be substituted with other components of similar function and amounts can vary accordingly.

TABLE 3
Media formulation used to promote T cell differentiation (Stage 3)

| Reagent | Concentration | |
|---|---|---|
| Ascorbic acid | 60 | uM |
| 2-Mercaptoethanol | 24 | uM |
| SCF | 0.02 | μg/mL |
| Flt3L | 0.02 | μg/mL |
| TPO | 0.02 | μg/mL |
| IL-7 | 0.02 | μg/mL |
| IL-3 | 0.01 | μg/mL |
| TNFa | 0.005 | μg/mL |

Base media is Iscove's Modified Dulbecco's Medium with 20% BIT 9500 Serum Substitute, 0.05% human Low-Density Lipoproteins

### Example 1: A Three Step Approach for Feeder-Free T Cell Differentiation from Pluripotent Stem Cells

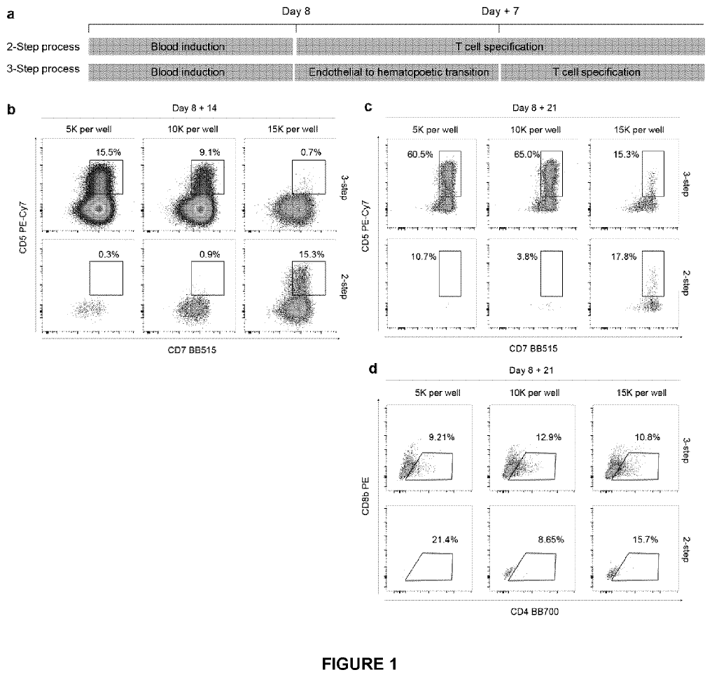

A schematic diagram of the two (2) step prior art process without EHT and the three (3) step process with EHT of the present invention is shown in FIG. 1a. FIG. 8 is a schematic overview of key developmental stages during differentiation from pluripotent stem cells to T cells in accordance with the three (3) step process of the present invention

In the three-step process of the present invention, pluripotent stem cells aggregate, and the aggregated cells are directed to become blood progenitors and then cultured on a cell culture substrate functionalised with recombinant DLL4 and VCAM1 in a media designed to support endothelial to hematopoietic transition (EHT). Next, the cells are grown in media optimised for T cell differentiation on a cell culture substrate functionalised with recombinant DLL4 and VCAM1.

Representative flow cytometry analysis of iPS11 cells subjected to either a 2-step or 3-step differentiation protocol after 22 days are shown in FIG. 1 b. Gated cells express CD5 and CD7, indicative of T cell progenitors. FIGS. 1 c and d are representative flow cytometry analysis of iPS11 cells subjected to either a 2-step or 3-step differentiation protocol after 29 days. FIG. 1 c shows that gated cells express CD5 and CD7, indicative of T cell progenitors. FIG. 1 d shows that gated cells are immature CD4 single positive T cell progenitors.

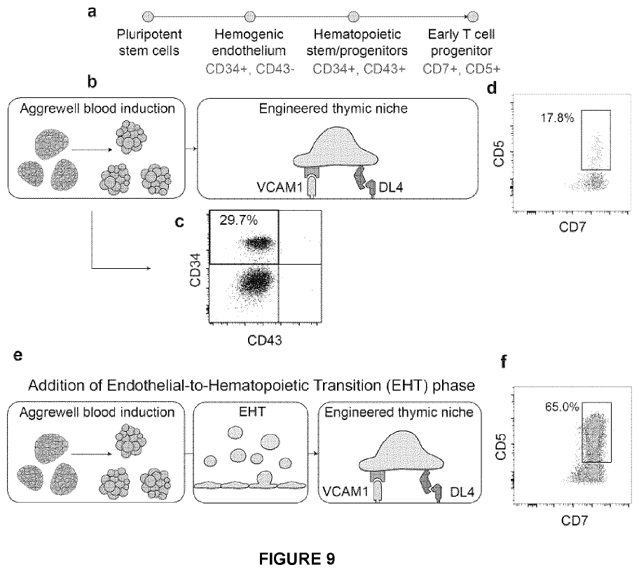

FIGS. 1 b-d and FIGS. 9 d and e and f, illustrate that the addition of a culture stage that supports endothelial to hematopoietic transition (EHT) improves progenitor T cell output, ie. 17.8% for two-step process (FIG. 9d) versus 65% for the three-step process of the present invention (FIG. 9f).

By way of background, FIG. 9a is a schematic overview of developmental stages between pluripotent stem cells and T cell progenitors and their associated immunophenotype (i.e. cell surface marker expression).

FIG. 9c is a representative flow cytometry analysis of the cells generated during the aggrewell blood induction (phase 1) portion of the protocol.

FIG. 9d is a representative flow cytometry of the cells generated after enriching for CD34+ cells following aggrewell blood induction and subsequently culturing the enriched cells for 14 days in T cell differentiation media (2-step protocol).

FIG. 9e is a schematic overview of a 3-step differentiation protocol where FIG. 9f is a representative flow cytometry of the cells generated after enriching for CD34+ cells after aggrewell blood induction and subsequently cultured for 7 days in media formulated to support endothelial to hematopoietic transition (EHT) in the presence of DLL4 and VCAM1 and then for 7 additional days in T cell differentiation media (3-step protocol).

### Example 2: A Three-Step Approach Improves T Cell Differentiation from Pluripotent Stem Cells

FIG. 2 illustrates a yield of CD5+, CD7+ T cell progenitors produced after 22 days using either a 2-step or 3-step differentiation process. Error bars reflect standard deviation of 3 independent replicates. b) Yield of CD5+, CD7+ T cell progenitors produced after 29 days using either a 2-step or 3-step differentiation process. Error bars reflect standard deviation of 3 independent replicates. c) Yield of CD4ISP T cell progenitors produced after 29 days using either a 2-step or 3-step differentiation process. Error bars reflect standard deviation of 3 independent replicates.

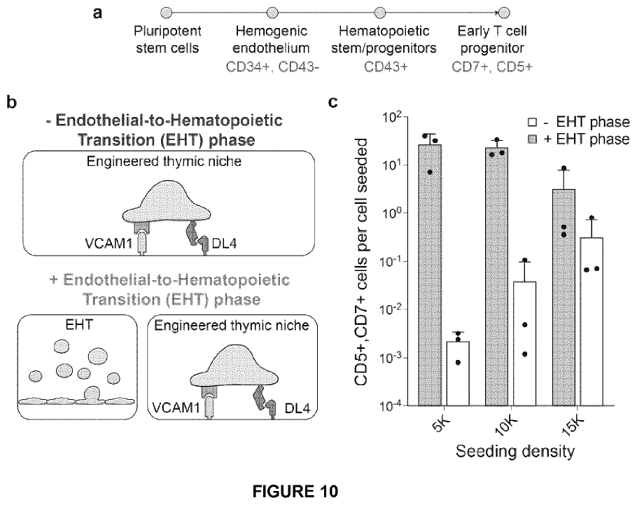

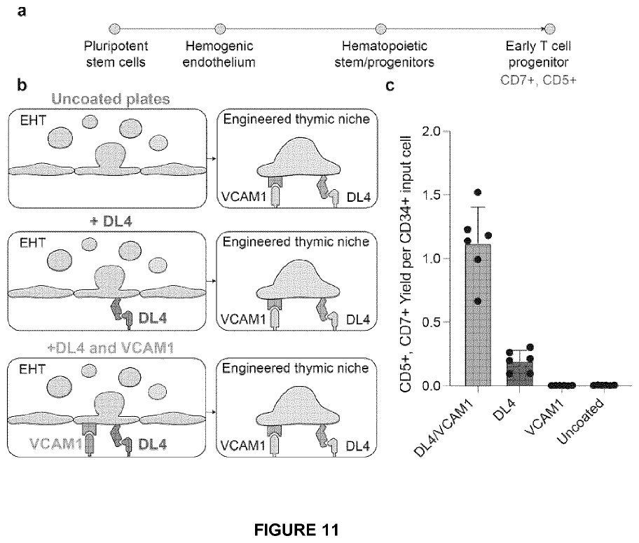

One can further see from FIGS. 2 a-c and FIG. 10c that the T cell progenitor output across cell seeding densities is better (or much enhanced) in the three-step EHT process of the present invention over the 2-step process, the figure illustrating quantification of the yield of CD5+, CD7+ T cell progenitors (early T cell progenitors) between the 2-step and 3-step protocols and across seeding densities as determined by flow cytometry and FIG. 2(c) illustrating the generation of later stage T cell progenitors (CD 4+). Notably, after 22 days of differentiation the yield of early T cell progenitors is more than 25 times greater in the 3 step-protocol in present invention compared to the 2-step protocol. After 29 days, the yield of CD5+, CD7+ T cell progenitors is 85 times greater in the 3-step protocol compared to the 2-step protocol. By way of background FIG. 10 a. is a schematic overview of developmental stages between pluripotent stem cells and T cell progenitors and FIG. 10 b is a schematic overview and of a 2-step and 3-step differentiation protocol.

### Example 3: A Three Step Approach Generates T Cell Progenitors with a Distinct Transcriptional Profile

FIG. 3 illustrates a) Single cell RNA sequencing analysis performed on T cell progenitors generated from iPS11 in vitro. Unsupervised leiden clustering reveals distinct gene expression profiles for cells produced using the 3-step protocol compared to cells produced using the 2-step protocol. b.) Top 20 overexpressed genes in 3-step protocol (left) and 2-step protocol (right) plotted by Z-score (wilcoxon rank-sum test). c, d and e) Violin plots comparing expression levels between protocols for house-keeping genes (c) and a selection of genes that are overexpressed in the 3-step (d) and 2-step (e) protocols. Cells generated in the 3-step protocol, when compared to cells generated using the 2-step protocol, express substantially higher levels of IGLL1, indicative of enhanced B cell potential and higher expression of the chemokine receptor CXCR4 which is expected to lead to enhanced cell migration and bone marrow and thymic engraftment. The cells generated in the 3-step protocol also express higher levels of the HLA class 1 genes HLA-A, HLA-B and B2M.

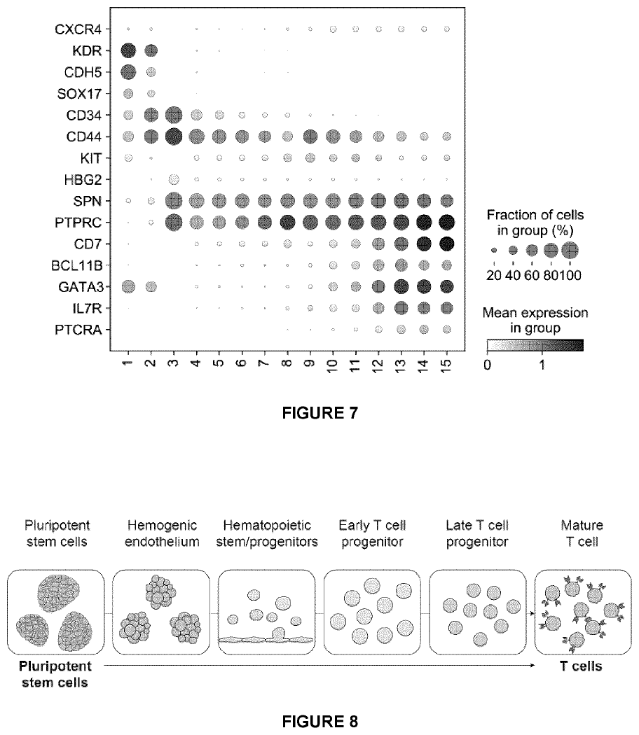

FIG. 7 illustrates gene expression changes over time during endothelial to hematopoietic transition and progenitor T cell differentiation. New endothelial to hematopoietic cultures were initiated from cryopreserved CD34+ cells on DLL4 and VCAM1 every day for 14 days. After 7 days in media designed to support endothelial to hematopoietic transition, cells were passaged into T cell differentiation media in the presence of DLL4 and VCAM1. After 14 days, cells from each time point were collected and subjected to single-cell RNA sequencing. Expression of genes associated with endothelial, hematopoietic and T cell identity are plotted over time. Time point 1 is the initial CD34+ population and time point 15 are cells that have gone through 7 days of endothelial to hematopoietic culture and 7 days of T cell differentiation. These cells express the genes expressed by primary human hemogenic endothelial cells including CDH5, CXCR4 and KDR and subsequently express genes expressed by primary human hematopoietic stem and progenitor cells such as CD34, SPN, PTPRC and subsequently express genes that are known to be expressed by primary human T cell progenitors such as CD7, BCL11B, IL7R and PTCRA.

### Example 4: The Three Step Protocol Produces Progenitors Capable of Maturing into Conventional, and TCR Engineered T Cells

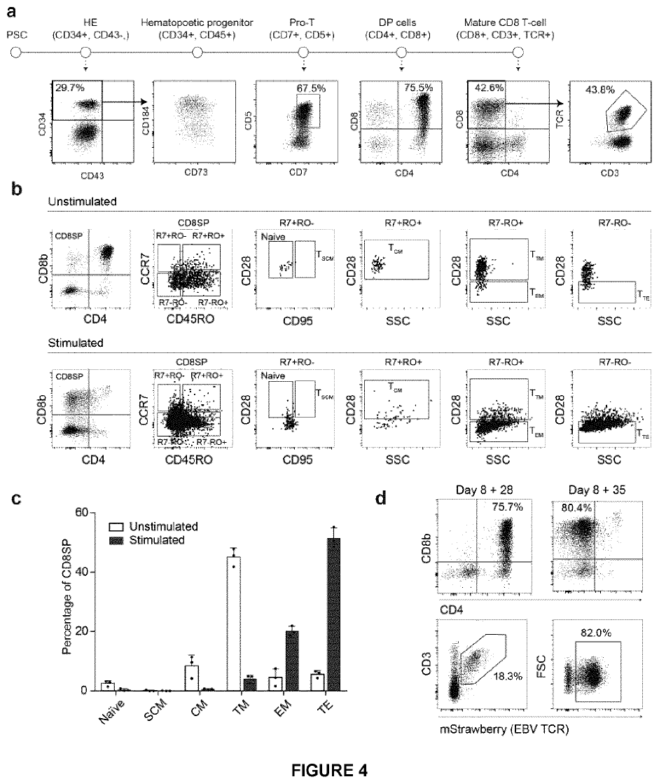

FIG. 4 a) illustrates a flow cytometry analysis that confirms that pro T cells generated from iPS11 using the 3-step differentiation protocol can continue to mature into CD8+, αβTCR+, CD3+ T cells using defined differentiation conditions. b) Flow cytometry analysis of effector and memory phenotypes of CD4+, CD8+ double positive (DP) cells and CD8 single positive (SP) T cells generated by stimulating DPs with αCD3, αCD2, αCD28 beads that are used to provide a stimulatory signal mimicking thymic positive selection and promoting the maturation from DP cells to SP cells. Results are quantified in (c) and demonstrate that stimulated cells tend to display an effector phenotype while unstimulated cells display a range of naïve, effector and memory phenotypes. d) T cells engineered with a lentiviral vector encoding an αEBV T cell receptor and an mStrawberry reporter are able to mature into DP and SP T cells.

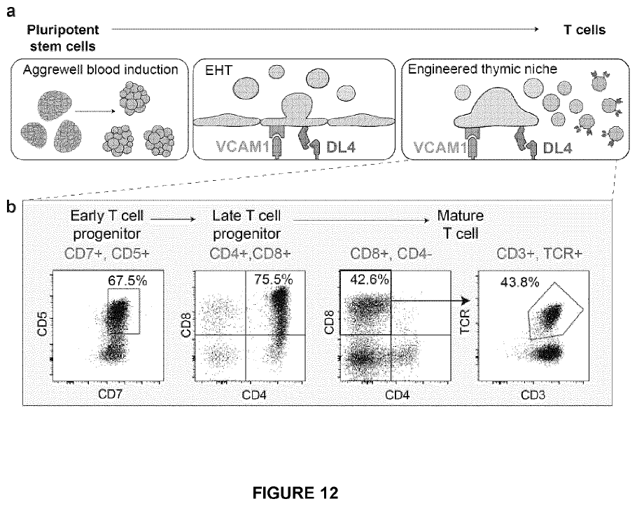

Further the inventors have shown that blood (hematopoietic/stem) progenitors generated in the presence of DLL4 and VCAM1 can differentiate to mature T cells with good frequencies and yields. See FIG. 12b. FIG. 12 a is a schematic overview of 3-step differentiation process, where as FIG. 12b are representative flow cytometry results demonstrating that the blood progenitors generated in media that supports endothelial to hematopoietic transition in the presence of DLL4 and VCAM1 can progressively mature into early T cell progenitors, late T cell progenitors and mature T cells.

### Example 5: Production of T-Competent Blood Progenitors is Notch Dependent and is Enhanced by the Combination of DL4 and VCAM1

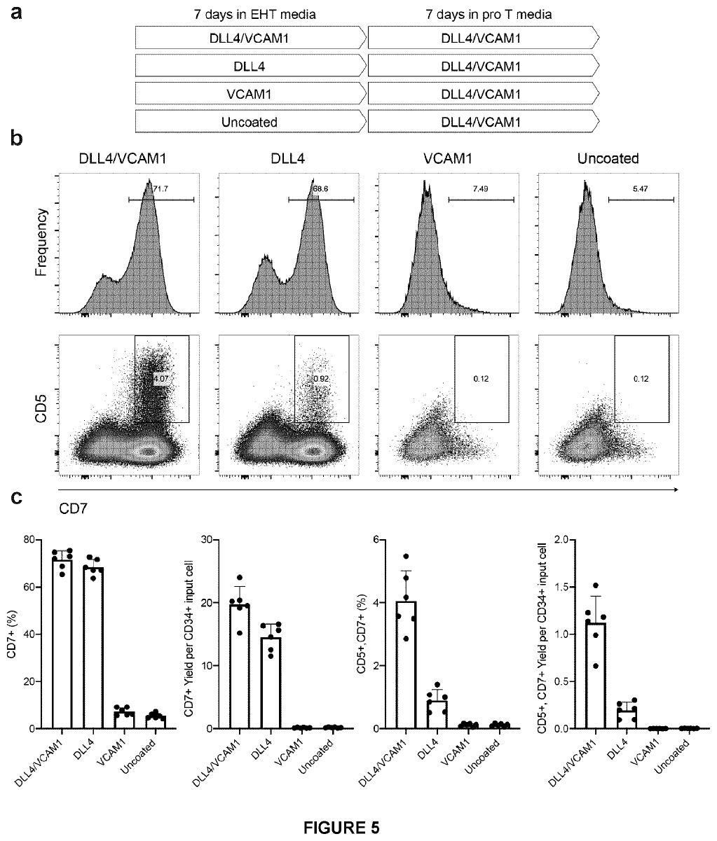

The inventors performed the three-step process using various ligand combinations in the EHT phase (uncoated, DLL4 alone, VCAM1 alone and DLL4 and VCAM. The ligands in the pro T media phase remained consistent (DLL4 and VCAM1) (see FIG. 5a). Results are shown in FIGS. 5b, 5c and FIG. 11c.

More particularly, FIG. 5: a) Experimental schematic. Cells were cultured in media designed to support endothelial to hematopoietic transition on uncoated culture plates, plates coated with Fc-DLL4, with Fc-VCAM1 or both proteins for 7 days. All conditions were subsequently passaged on to plates coated with both Fc-DLL4 and Fc-VCAM1 proteins in the presence of media that supports pro T cell differentiation. b) Representative flow cytometry analysis of CD7+ and CD5+, CD7+ T cell progenitors generated in each condition at day 14. c) Quantification of the frequency and yield of T cell progenitors from (b) across conditions (n=6 technical.

FIG. 11C illustrates the results of quantification of the yield of CD5+, CD7+ T cell progenitors after undergoing endothelial to hematopoietic transition in the presence of the indicated immobilized proteins, or an uncoated control surface.

### Example 6: Hematopoietic Stem and Progenitor Cells Produced in the Presence of DL4 and VCAM1 have a Definitive Transcriptome and Multilineage Potential

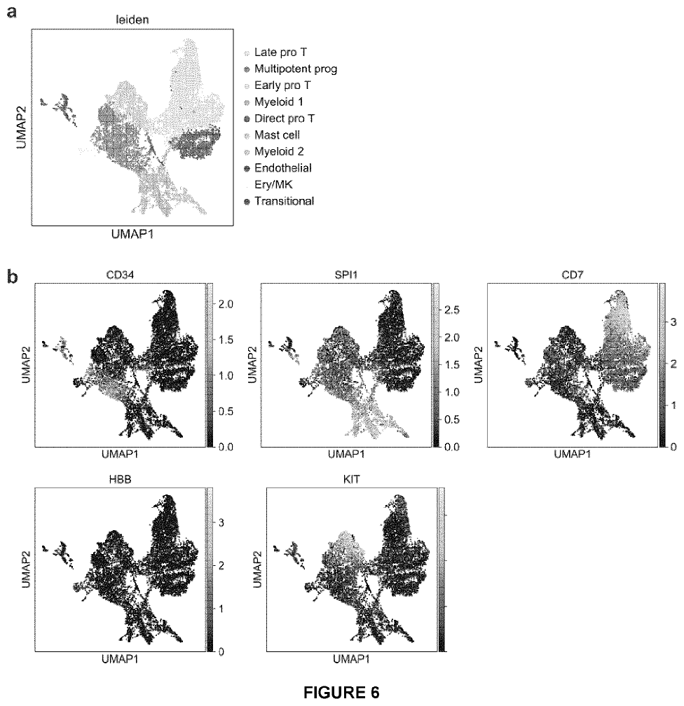

FIG. 6: a) Universal Manifold Approximation and Projection (UMAP) and unsupervised leiden clustering analysis of single cell RNA sequencing (scRNA-seq) data obtained for cells sampled at each day of the EHT stage and for 7 days of the subsequent T cell differentiation culture. Ery/MK=erythroid megakaryocyte progenitors. Endothelial and hematopoietic cell types were identified on the basis of marker gene expression shown in (b). Notably, the generated cells display both lymphoid and myeloid potential and produce erythroid progenitors that express definitive globin genes including HBB. Hemogenic endothelial cells and blood progenitor cells express CD34, Ery/Mk progenitors express the hemoglobin gene HBB, mast cell progenitors express KIT, myeloid cells express high levels of the transcription factor SPI1 and T cell progenitors express CD7.

### Example 7: An Engineered Niche Comprising Immobilized DLL4 and VCAM1 Supports Highly Efficient Development of T Competent Hematopoietic Progenitors from hPSC

The inventors herein illustrate an efficient, chemically defined process and system for differentiating conventional T cells from human pluripotent stem cells (hPSCs). The process and system is compatible with clinical applications and user-customisation is herein illustrated. One important bottleneck in T cell differentiation from hPSC is producing hematopoietic stem/progenitor cells with T lineage potential.

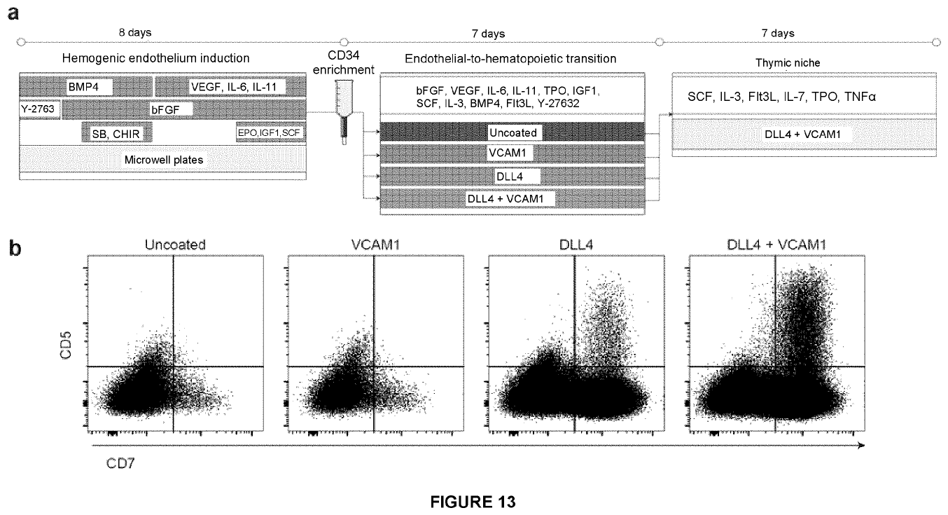

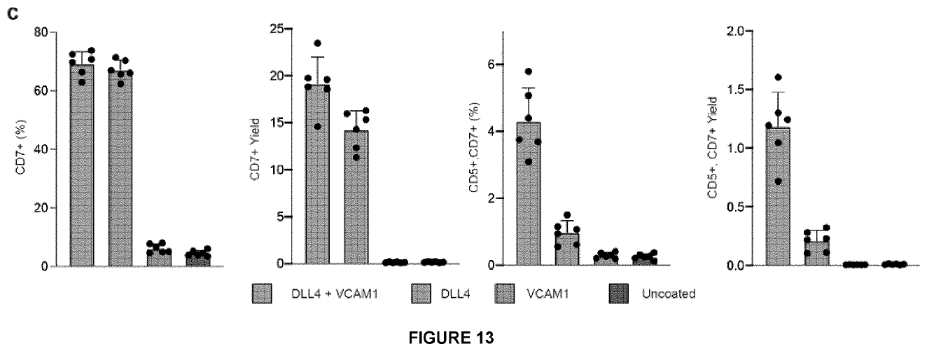

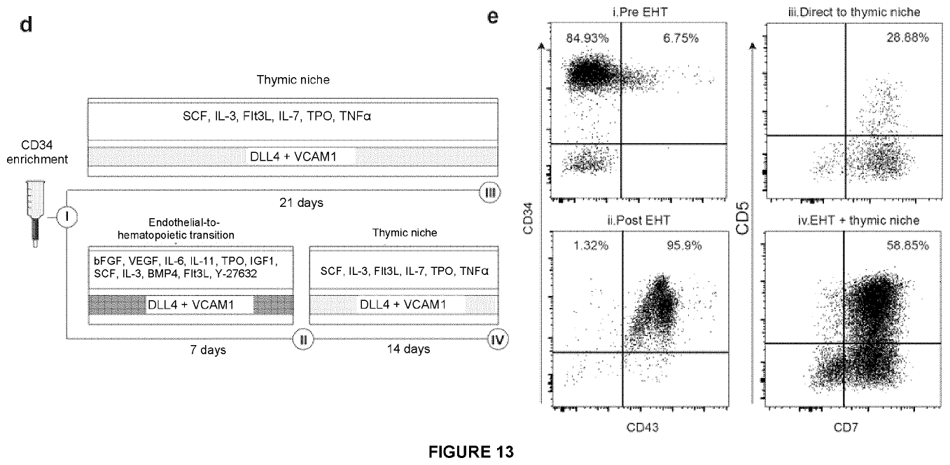

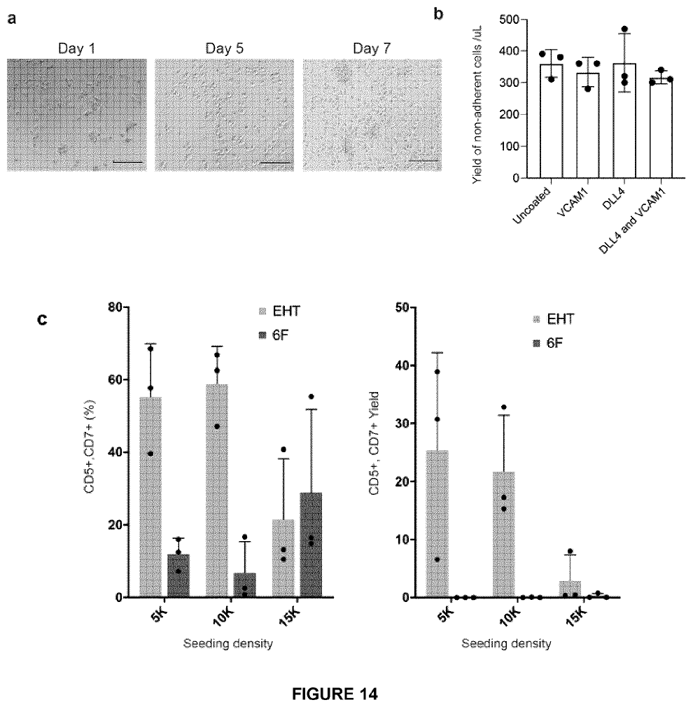

Hemogenic endothelial (HE) cells, the developmental precursors of hematopoietic stem cells, were generated by aggregating hPSC into 3D structures by centrifugation in microwell plates. The aggregates were subjected to a step-wise series of media formulations known to specify mesoderm, and subsequently definitive haemato-endothelial identity. The resulting aggregates were dissociated and CD34+ cells were separated to enrich for HE. The CD34+ cells were placed onto an uncoated tissue culture surface in media formulated to promote endothelial-to-hematopoietic transition (EHT) (FIG. 13a). Morphological transition from adherent, endothelial like cells, to non-adherent spherical cells was observed (FIG. 14a).

Next, the cells were assayed to see whether these non-adherent hematopoietic cells were capable of differentiating into T cell progenitors. The cells were transferred to plates coated with immobilized DLL4, a notch ligand capable of promoting T lineage specification, and VCAM1, a cell adhesion molecule (FIG. 13 a). The cells were cultured in media capable of generating CD5+, CD7+ T cell progenitors from cord-blood stem cells, however almost no CD5+, CD7+ cells were detected from hPSC derived hematopoietic cells (FIG. 13 b,c). In fact, very low numbers of CD7+ cells were observed, an earlier and less committed progenitor (FIG. 13b,c).

Next it was attempted to obviate the need for feeder cells by including immobilized recombinant DLL4 during the EHT culture phase (FIG. 13a). When the HSPC generated on DLL4 were transferred into the thymic niche, a striking improvement in progenitor T cell differentiation compared to HSPC generated on uncoated plates was observed. The presence of DLL4 during EHT increased the median frequency of CD7+ cells generated to 66.51% compared to 4.22% in the uncoated condition (P=0.002, Mann Whitney test, FIG. 13c). DLL4 increased the yield of CD7+ cells produced per input cell over 90-fold compared to the uncoated control (FIG. 13c). Encouragingly, DLL4 led to the emergence of a distinct population of the later stage CD5+, CD7+ T cell progenitors, a population that was nearly absent from the uncoated condition (FIG. 13b). These data demonstrate that the addition of recombinant DLL4 during EHT drastically improves the production of hematopoietic cells with T cell potential from hPSCs.

### Progenitor T Cell Output was Surprisingly Improved Even Further Using the Cell Adhesion Protein VCAM1 to Increase Notch Signalling During EHT and Thus Increase T Cell Potency.

VCAM1 in combination with DLL4 during EHT resulted in a dramatic increase in downstream production of CD5+, CD7+ T cell progenitors (FIG. 13b). VCAM1 and DLL4 together increased both the frequency (4.04 fold, P=0.0022, Mann Whitney test) and yield (5.73 fold, P=0.0022, Mann Whitney test) of CD5+, CD7+ cells compared to DLL4 alone (FIG. 13c). VCAM1 alone did not substantially alter progenitor T cell production compare to the uncoated control (FIG. 13b,c), suggesting that it acts cooperatively with DLL4 to enhance the differentiation. This effect cannot be attributed to an increase in the number of non-adherent hematopoietic cells produced during EHT as this number did not differ substantially between coating conditions (FIG. 14 b).

### The EHT Phase Improved the Yield of CD5+, CD7+ T Cell Progenitors.

It was tested as to whether T cell progenitor yields are improved by this EHT culture phase, or if CD34-enriched cells harvested from aggregates can be placed directly into the engineered thymic niche (FIG. 13d). It was found that the CD34+ cells harvested from the day 8 aggregates were largely CD43−, indicating that they have not yet established a hematopoietic identity (FIG. 13e). In contrast, >95% of the non-adherent cells harvested after EHT co-expressed CD34 and CD43 (FIG. 13e). Consistent with this transition to hematopoietic identity, the EHT phase improved the yield of CD5+, CD7+ T cell progenitors by 70-fold compared to placing cells directly into the thymic niche (FIG. 13e, FIG. 14c). The EHT phase significantly improved T cell progenitor yield across multiple cell seeding densities (suppX, P=0.001, two-way ANOVA).

As such, an engineered signalling environment comprising recombinant DLL4 and VCAM1 and an appropriate chemically defined, serum-free media is sufficient to support emergence of HSPC with robust T lineage potential. Further, it was shown that EHT worked well across densities, suggesting that culture conditions, rather than paracrine signals were driving their cell fate whereas direct to proT was heavily dependent on seeding density suggesting a reliance on paracrine factors.

It was Also Shown that DLL4 Skews hPSC Derived Hematopoietic Stem and Progenitor Cell Fate Distributions and the Addition of Plate-Bound DLL4 and VCAM1 During EHT Markedly Impacted the Ability of PSC-Derived HSPC to Differentiate Towards the T Lineage.

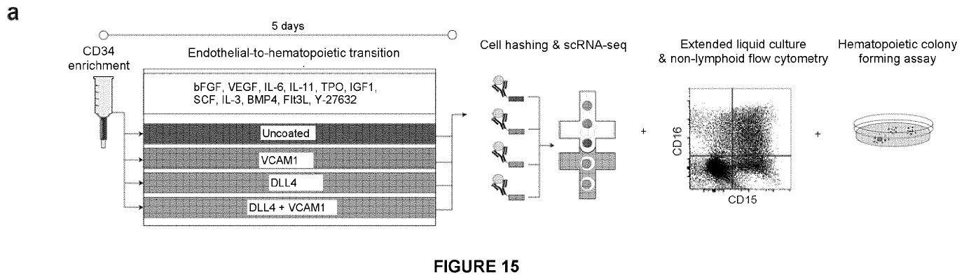

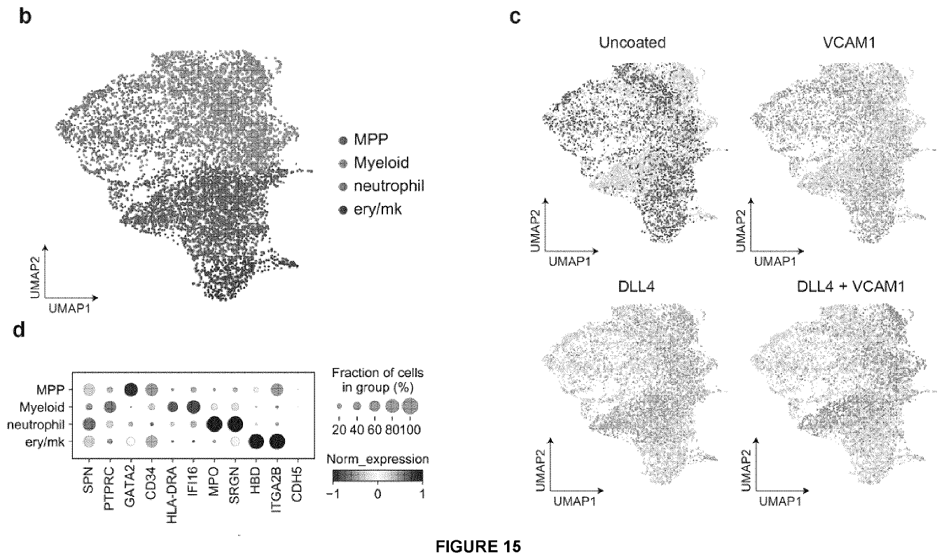

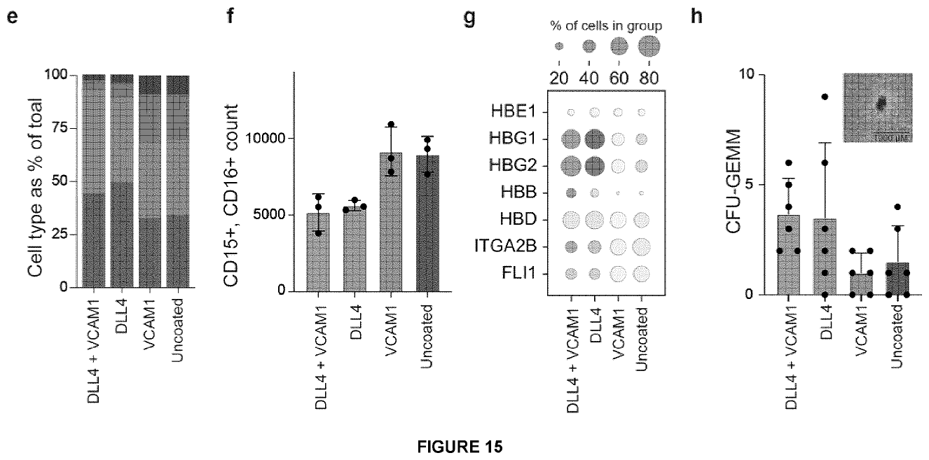

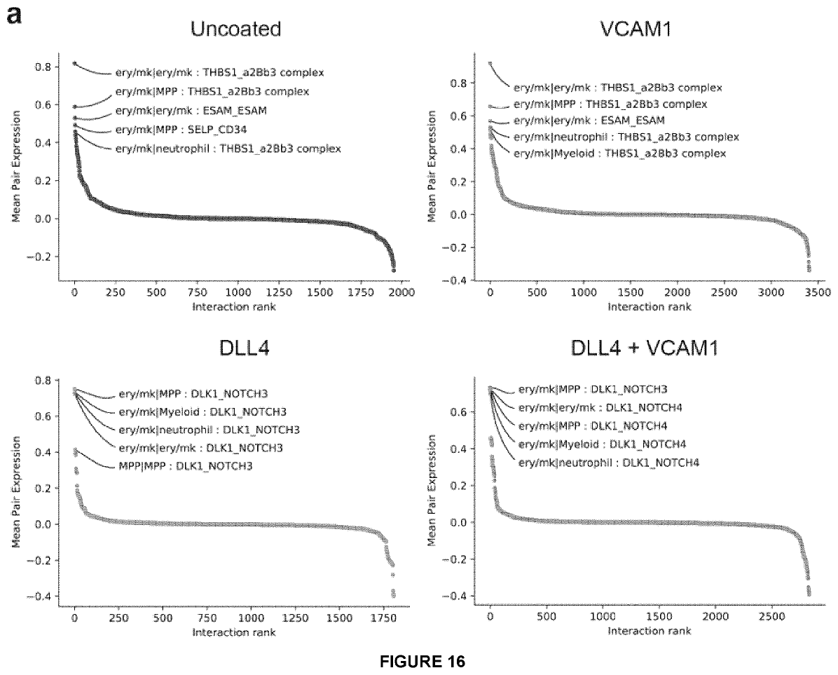

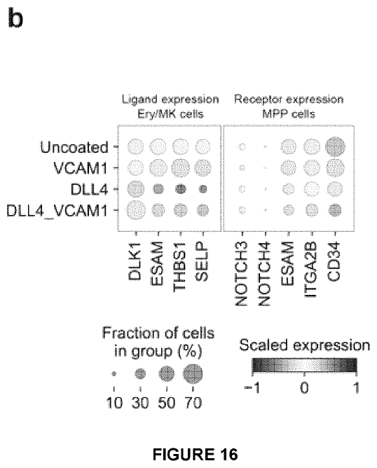

In the following examples, single cell RNA-sequencing was used to compare the hematopoietic stem/progenitor cells (HSPC) made during EHT in the presence or absence of DLL4 and VCAM1 (FIG. 15, 17, 18). It was shown that adding DLL4 during EHT increases expression of hemoglobin genes associated with definitive hematopoiesis. DLL4 during EHT decreases the frequency of neutrophil progenitors as measured by scRNA sequencing. The finding was confirmed by flow cytometry.

A droplet-based single cell RNA sequencing (scRNA-seq) was used to understand how these two proteins altered the HSPC transcriptional composition. Non-adherent cells generated on uncoated plates, plates coated with DLL4 or VCAM1 alone, or coated with both proteins were compared. Following doublet and dead-cell removal, 7,589 cells were obtained (FIG. 15a, b) including at least 1,500 from each of the four coating conditions (FIG. 15b,c). Four (4) sub-types of HSPC were identified on the basis of marker gene expression; SPN+, CD34+ hematopoietic stem cells/multipotent progenitors (HSC/MPP), HLA class II+, IFI16+ myeloid progenitors, HBD+, ITGA2B+ erythroid/megakaryocyte progenitors and SRGN+, MPO+ neutrophil progenitors (FIG. 15d). Most cells expressed the hematopoietic gene SPN and the endothelial genes KDR and CDH5 were nearly absent, consistent with successful transition from an endothelial to hematopoietic transcriptional program (FIG. 15d).

Coating conditions during EHT shifted the relative proportions of the resulting HSPC sub-types (FIG. 15e). The presence of DLL4 caused an increase in the frequency of HSC/MPP, decreased the occurrence of both erythroid/megakaryocyte progenitors and strongly reduced the abundance of neutrophil progenitors compared to VCAM1 alone or the uncoated control (FIG. 15e). Upon extended liquid culture, we confirmed that DLL4 reduced the output of CD15+, CD16+ neutrophils (FIG. 15f).

Within the erythroid/megakaryocyte progenitor population, it was observed that DLL4 reduced expression of the megakaryocyte transcription factors ITGA2B and FLI1, and increased expression of haemoglobin genes associated with erythroid specification (FIG. 15g). Extended liquid culture verified that DLL4 increased the ratio of CD235a+ erythroid cells to CD41+ megakaryocyte cells.

Notch signalling during EHT in vitro has been reported to promote emergence of HSPC comparable to a later stage in human ontogeny as evidenced by a switch in globin gene expression from embryonic to foetal. Indeed, upon addition of DLL4 increased expression of the foetal globin genes HBG1 and HBG2 and the adult globin gene HBD (FIG. 15g) was observed. When VCAM1 and DLL4 were added together, a further increase in expression of the adult globin HBB and decrease in the embryonic gene HBE1 was detected (FIG. 15g).

Colony forming assays demonstrated that DLL4 increased the frequency of the primitive colony forming unit, CFU-GEMM by 2.9 fold (FIG. 15h), (P=0.013). DLL4 drives a shift in HSPC cell composition, including a reduction in neutrophil output, higher ratios of foetal and adult to embryonic haemoglobin expression and an increase in the frequency of multipotent CFU-GEMM. Thus, DLL4 during EHT increases production of CFU-GEMM, a highly multipotent type of hematopoietic progenitor (FIG. 15h).

### DLL4 and VCAM1 Alter HSC/MPP Gene Expression Programs and Cooperatively Activate Notch Signalling

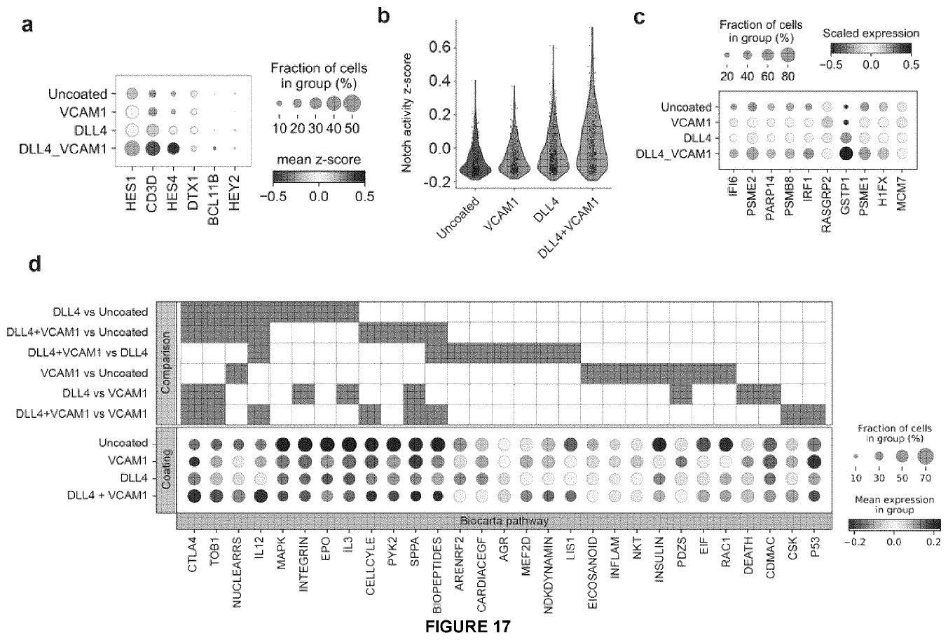

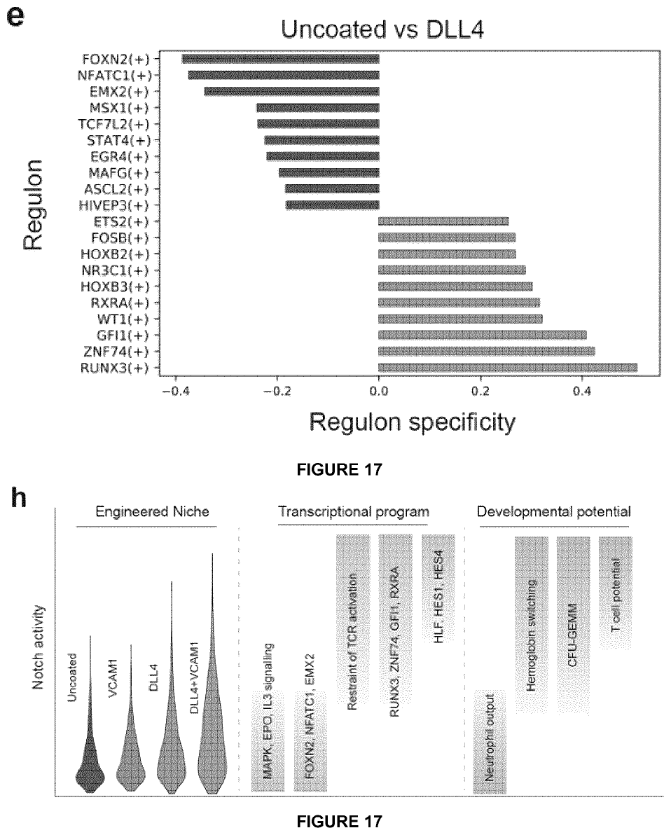

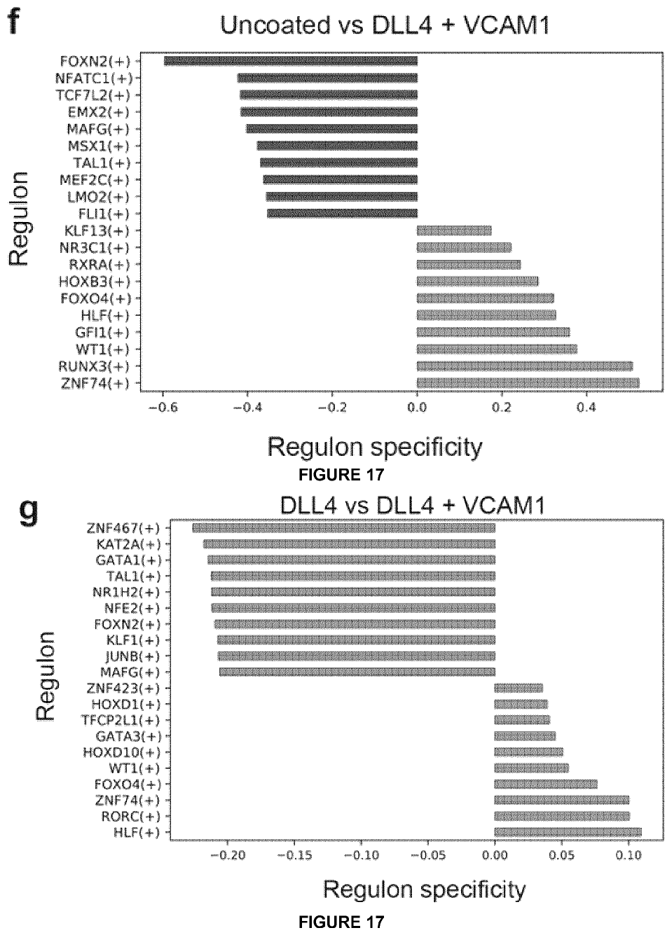

The impact of engineered signalling environment on the transcriptional identity of HSC/MPP produced during EHT was examined. It was shown that the presence of DLL4 increased the frequency and magnitude of expression of known downstream targets of Notch signalling (FIG. 17a,b). In a separate developmental context, it was shown that the cell adhesion molecule VCAM1 can enhance DLL4-mediated notch signalling. VCAM1 alone did not substantially alter expression of notch targets (FIG. 17b). In combination with DLL4, VCAM1 drastically increased expression of HES1, CD3D, HES4, DTX1, BCL11B and HEY2 compared to DLL4 alone (FIG. 17a, b). During EHT, VCAM cooperates with DLL4 to promote high levels of notch activity in HSC/MPP.

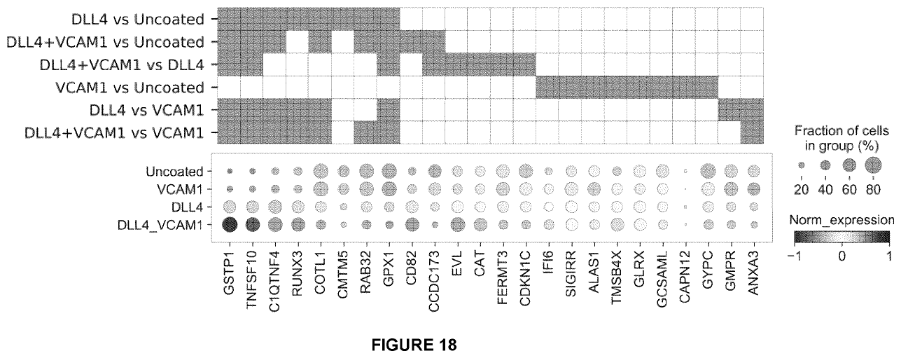

An unbiased exploration of the impact of coating conditions on MPP/HSC gene expression was conducted (FIGS. 17c, d, and 18). Increasing Notch signalling, differential expression analyses revealed that VCAM1 also altered expression of EVL and FERMT3, genes involved in cell adhesion and cytoskeletal polymerisation (FIG. 18), and increased activity of the interferon induced protein IF16 and multiple members of interferon-induced immunoproteasome, PSME2, PSMB8 and PSME1 (FIG. 17c). These changes in interferon responsive genes were observed, both when comparing VCAM1 to the uncoated control, and when comparing VCAM1 and DLL4 to DLL4 alone (FIG. 17c). Consistent with increased expression of interferon and inflammatory genes, pathway enrichment analysis confirmed that VCAM1 caused a significant increase in inflammatory pathway activity, both in the presence and absence of DLL4 (FIG. 17d).

The studies indicated that DLL4 drove an increase in several pathways associated with restraining T cell activation including CTLA4, TOB1 and CSK. These effects were magnified by the addition of VCAM1. DLL4 reduced activity of cell cycle and P53 pathways and increased activity of the death pathway. These alterations in transcriptional state are consistent with a model whereby notch signalling promotes emergence of an HSC/MPP population that is primed to undergo TCR-mediated selection during T cell differentiation.

SCENIC analysis revealed regulons (transcription factors and their downstream targets) that were specifically upregulated in each engineered signalling environment (FIG. 17e,f,g). Nearly all of the regulons that were most strongly activated by DLL4 have a known role in blood emergence and T cell differentiation in human or mouse (FIG. 17e). RUNX3 contributes to HSC maintenance and T cell differentiation. GF11 and FOSB are important drivers of endothelial to hematopoietic transition The homeobox gene HOXB3 is expressed in both uncommitted HSCs and during T cell development. WT1 is important for survival and maintenance HSC/MPP and downregulated in differentiated progeny. The DLL4-mediated increase in RXRA regulon activity merits future investigation given the complex role of retinoic acid signalling during the emergence of HSCs from hPSCs. ZNF74 was also amongst the regulons most strongly upregulated by DLL4 and has no previously documented role in haematopoiesis (FIG. 17e,f).

Notably, when added alongside DLL4, VCAM1 increased HLF and GATA3 regulon activity (FIG. 17g). A recent report suggests that HLF expression is the most selective identifier of engraftable HSC reported to date. In addition to its appreciated role in human T cell development, Gata3 helps maintain the pool of LT-HSC in. Thus DLL4 and VCAM1 during EHT promote activity of hematopoietic transcription factors such as HLF and GATA3 (FIG. 17).

Collectively, these data demonstrate that introducing DLL4 and VCAM1 during EHT positively shifts the distribution of notch pathway activity in the resulting HSC/MPP (FIG. 17b, h). This shift in notch activity is associated with marked changes in transcription factor and pathway activity and alters cell fate distribution, with intermediate levels of notch activity driving haemoglobin switching and suppressing neutrophil output and high notch activity unlocking T cell potential (FIG. 17h).

### Model Guided Media Optimisation Enables Efficient Production of CD8+, CD4−, CD3+, TCRαβ+ T Cells

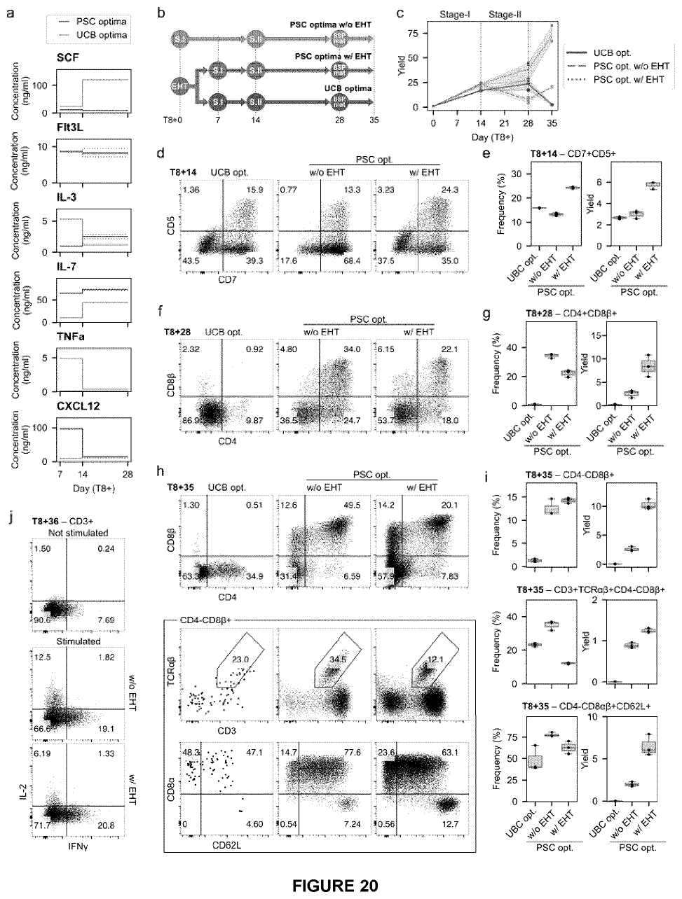

After demonstrating that the addition of DLL4 and VCAM1 during EHT is sufficient to support robust differentiation of CD7+, CD5+ T cell progenitors, it was sought to progress these cells towards a more mature CD4+, CD8+ phenotype. (FIG. 20 b-g). PSC-derived hematopoietic progenitors were transferred to a chemically defined thymic niche in serum-free media supplemented with a cytokine composition that we previously optimised for generating CD4+, CD8+ cells from umbilical cord blood (FIG. 20b). This proved to be inefficient (FIG. 20f,g). It was hypothesized that PSC-derived hematopoietic progenitors may require distinct cytokine concentrations to efficiently differentiate into T cells.

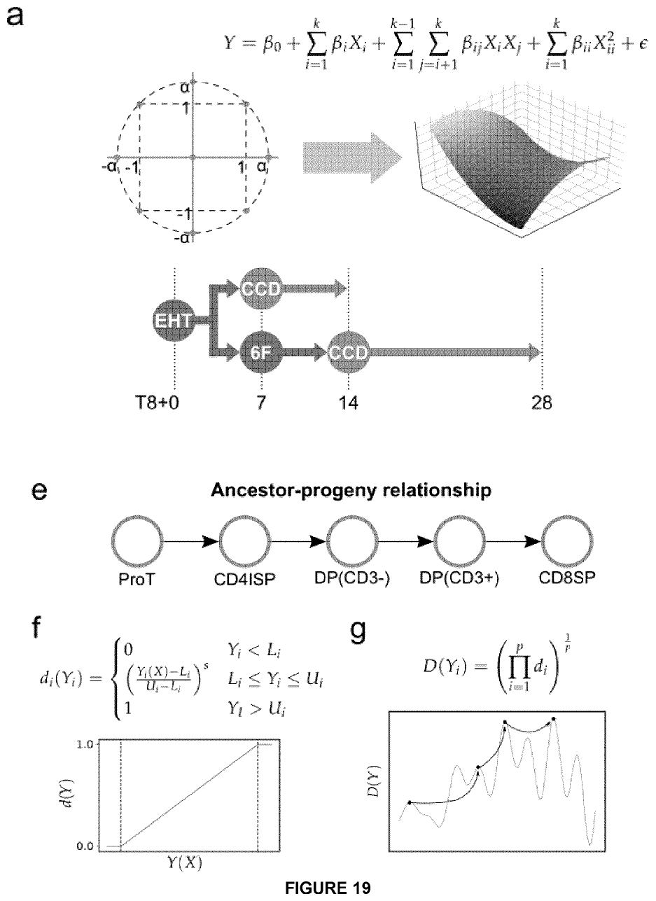

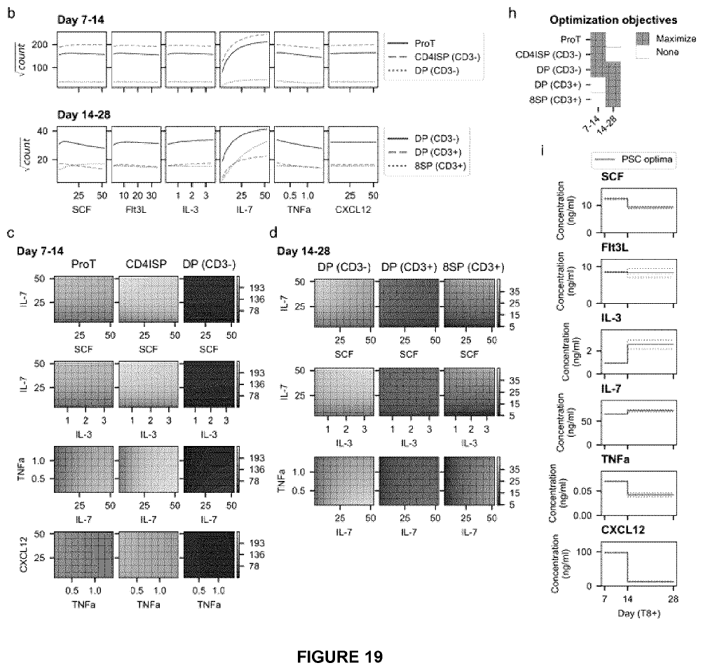

To address this limitation and improve PSC-derived T cell production, a multi-stage modelling approach was used to optimise the cytokine concentrations in the inventors' defined T cell differentiation media. A central composite design (CCD) to screen 5 concentrations of 6 different cytokines over two stages of development was implemented, a 7-day early progenitor T cell stage followed by a 14-day maturation stage (FIG. 20). The yield of five successive T cell developmental phenotypes was measured, CD5+, CD7+ progenitors (proT), CD4+, CD8−, CD3− immature single positive (CD4ISP), CD4+, CD8+, CD3− double positive progenitors (DP, CD3−), CD+, CD8+, CD3+ (DP, CD3+) and CD3+, CD4−, CD8β+ (CD8SP). Relevant phenotypes were fit to polynomial dose response models for each cytokine at both time points (FIG. 19).

Over the 7-day early differentiation stage, proT, CD4ISP and DP, CD3+ output had a strong positive response to IL-7 concentration and a moderate positive response to CXCL12. Outputs of these populations exhibited a negative response to increasing TNFα concentrations. During the maturation stage, CD3− and CD3+DP as well as CD8SP responded positively to IL-7 and IL-3 and negatively to TNFα (FIG. 19). This CCD design also allowed the examination of multi-factor interactions (FIG. 19).

Optimal combination of cytokine concentrations for each differentiation stage were identified. A desirability score was developed whereby the geometric means of ProT, CD4ISP and CD3−, DP yields were combined for the early stage and CD3−, DP, CD3+, DP and CD8SP were combined for the maturation stage. Basin-hopping was applied to predict factor concentrations that would maximise desired phenotypes at each stage. After performing the optimisation from 25 random initialisations, the top 5 most desirable solutions were retained to calculate optimal cytokine concentrations for generating T cells from PSC derived hematopoietic progenitors.

These two newly optimised media formulations (PSC optima) were compared against the previously developed cord blood (CB)-optimised media (CB control, FIG. 20 a,b). PSC optima improved total cellularity compared to the CB control as early as day 14 and the magnitude of this effect was amplified over the course of the differentiation (FIG. 20c). Furthermore, the yield of desired cell types was drastically improved by the new media formulation. The PSC-optimised early stage media increased the abundance of CD7+, CD5+ proT cells two-fold compared to the CB control (FIG. 20d,e).

Following the maturation stage, PSC optima caused a striking 40-fold improvement in CD4+, CD8+ DP yields compared to the CB control (FIG. 20f,g). Non-specifically activated TCR signalling was conducted to simulate positive selection and quantified the yield of CD8SP, CD3+, TCRαβ+cells. The optimised media improved the yield by more than 150-fold compared to the CB control (FIG. 20h,i).

One limitation of previous in vitro differentiation methods, is that they tend to produce T cells with unconventional immunophenotypes. A common unintended product are cells that express a CD8αα homo-dimer, a characteristic feature of innate-like cells. PSC-derived T cells have previously been reported to lack robust expression of the adhesion molecule CD62L. The T cells generated using the optimised media of the present invention express the conventional CD8αβ heterodimer and robustly express CD62L (FIG. 20h,i). They produce IFNγ and IL-2 in response to non-specific stimulation (FIG. 20j).

The engineered EHT niche and optimised T cell differentiation media that we establish here each improve T cell production by orders of magnitude. Collectively, these improvements contribute to a highly efficient protocol that will aid both developmental studies and scalable production of T cell therapeutics.

In summary, when CD34+ cells were seeded directly into the optimised media in the presence of DLL4 and VCAM1, they generated appreciable quantities of mature T cells, albeit at reduced levels compared to cells first cultured in EHT media. Further, delayed kinetics of EHT system could likely boost yield even further by extending pro T/maturation stages. Also, the present inventors have shown that during ontogeny blood and T cell development are carefully orchestrated and dynamic process that take place in a series of distinct signalling niches.

In addition the data affirm that optimal differentiation outcomes require control over both the juxtracrine immobilised niche proteins, the concentration of soluble media factors, and the timing that they are administered. There is a striking difference achieved by tuning the dosage of each cytokine in the media (exact same factors as CB media, just altered levels enabled massive change in yield).

### Example 8: Optimized Cytokines Enhance T-Cell Development

As shown in FIGS. 19 and 20, the method of the present invention shows that optimizing cytokines in the media can enhance T-cell development. Table 4 illustrates an example of early and later stage media that can be used in some embodiments of the invention.

In this Example, the inventors optimized the downstream differentiation using media starting after the EHT step and going all the way until the development of mature T cells (FIGS. 19, 20 and 21).

The T cells made with this protocol can respond to activation by expressing effectors that are T cells are expected to make (FIGS. 20 and 21).

TABLE 4
Media formulation used in early and late stage T cell differentiation

| | |
|---|---|
| Component | Concentration |

Early T cell differentiation media

| IMDM | | |
|---|---|---|
| B27 minus vitamin A | 4% | |
| Pen/Strep | 0.50% | |
| 2-mercaptoethanol | 24 | uM |
| Ascorbic acid | 60 | uM |
| SCF | 12.37 | ng/mL |
| Flt3L | 8.61 | ng/mL |
| IL-3 | 0.97 | ng/mL |
| IL-7 | 65.25 | ng/mL |
| TNFa | 0.07 | ng/mL |
| CXCL12 | 97.4 | ng/mL |

Late stage T cell maturation media

| IMDM | | |
|---|---|---|
| B27 minus vitamin A | 4% | |
| Pen/Strep | 0.50% | |
| 2-mercaptoethanol | 24 | uM |
| Ascorbic acid | 60 | uM |
| SCF | 9.76 | ng/mL |
| Flt3L | 8.49 | ng/mL |
| IL-3 | 2.55 | ng/mL |
| IL-7 | 71.93 | ng/mL |
| TNFa | 0.04 | ng/mL |
| CXCL12 | 15.22 | ng/mL |

The invention is not limited to the particular media or staged media noted herein. For instance, the base media can be any suitable media designed and optimized for growing and/or culturing the particular cell type or known to support the particular cell growth or cells. In addition the other components could be substituted with other components of similar function and amounts can vary accordingly.

### Example 9: Three-Step +EHT Protocol Increased Frequency of CD34+ Cells with CD7+ Lymphoid Potential.—10 Fold Improvement

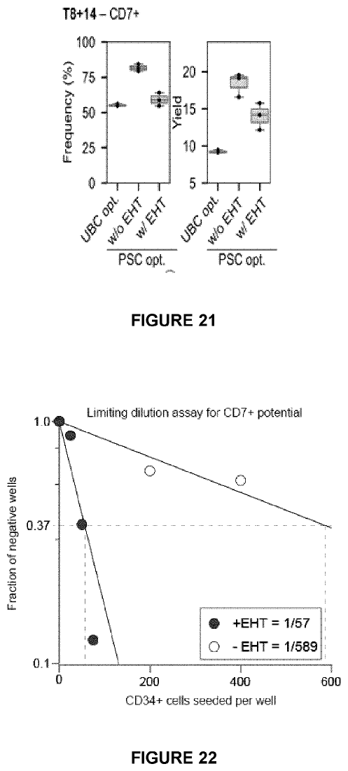

A limiting dilution analysis was conducted in order to assess whether the addition of the EHT phase increased the frequency of CD34+ cells that are able to give rise to CD7+ lymphoid progenitors. Results are shown in FIG. 22.

The cell numbers as indicated in FIG. 22 were seeded in 96 well plates and cultured for a total of 14 days (either 7 days EHT+7 days pro T differentiation media, or 14 days pro T differentiation media, according to the two-step and three-step protocols described in Example 1 (A three step approach for feeder-free T cell differentiation from pluripotent stem cells). Frequencies were modelled as the number of cells required to achieve a failure rate of 0.37 as this is the expected failure rate based on Poisson statistics when seeding 1 CD7 competent progenitor per well.

The frequency of 1/57 (57 cells to get one true T cell progenitor) for the three-step process (+EHT) compared to 1/589 for the two-step process (−EHT) (FIG. 22) demonstrates that the 3-step process of the present invention (+EHT) increases the frequency of CD34+ cells with CD7+ lymphoid potential by more than 10-fold compared to the two-step (−EHT) process. This is a greater than 20 fold increase against the previously published results of (Trotman-Grant et al, Trotman-Grant, A. C., Mohtashami, M., De Sousa Casal, J. et al. DL4-μbeads induce T cell lineage differentiation from stem cells in a stromal cell-free system. Nat Commun 12, 5023 (2021). https://doi.org/10.1038/s41467-021-25245-8) that lacks an explicitly EHT stage, which illustrated that only 1/1341 CD34+ cells were able to give rise to T-lymphoid progenitors.

### Example 10: Cells Generated Using the Three-Step (+EHT) Process and Optimized Cytokines have a Diverse T-Cell Receptor (TCR) Repertoire

For many applications, it is desired to generate a diverse T-cell population. To assess the diversity of the T cell population generated by the three-step (+EHT) process, PSC-derived CD34+ cells were subjected to EHT in the presence of DLL4 and VCAM1 as described in Example 8. Non-adherent hematopoietic cells were differentiated into T cells using the optimised conditions described in Example 8. After 21 days of T cell differentiation, 1×10⁶ cells per sample were pelleted at 400×g for 6 minutes and washed once with PBS. Genomic DNA was extracted using QuickExtract DNA Extraction Solution (Lucigen, QE9050) following the manufacturer's instructions and diluted in Tris-EDTA. TRB ImmunoSEQ survey resolution sequencing was performed by Adaptive Biotechnologies. TRB sequences from PSC-derived cells were compared to reference data previously acquired and reported in Edgar et al. (Edgar, J. M., Michaels, Y. S. & Zandstra, P. W. Multi-objective optimization reveals time- and dose-dependent inflammatory cytokine-mediated regulation of human stem cell derived T-cell development. npj Regen Med 7, 11 (2022). https://doi.org/10.1038/s41536-022-00210-1)

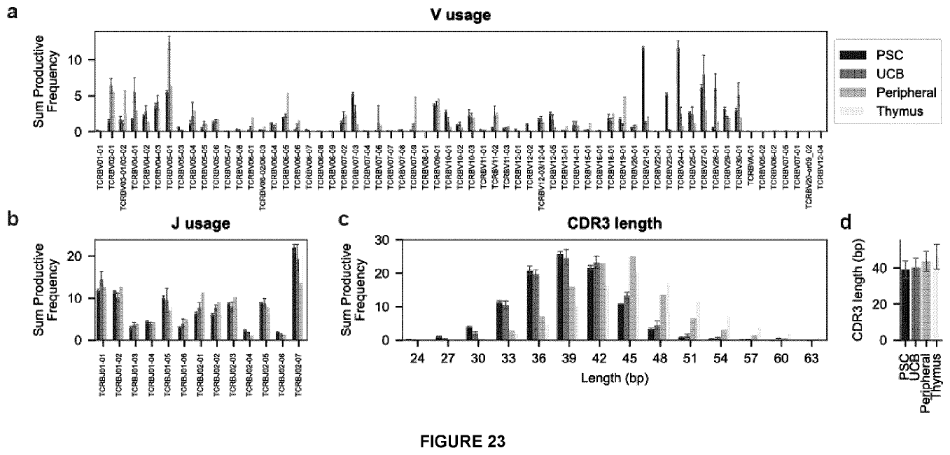

The results are illustrated in FIG. 23. The PSC-derived T cells produced by the method of the present invention expressed a diverse TCR repertoire with broad TCRV and TCRJ usage (FIGS. 23 a and b). While the TCR diversity of PSC-derived T cells was general comparable to T cells differentiated from umbilical cord blood and primary thymocytes, an increased usage of TCRBV21-01, TCRBV23-01 and TCRBV24-01 was observed (FIGS. 23 a and b). A previous analysis of TCRBV usage over developmental time showed enrichment for TCRBV24-01 in CD4−, CD8− DN progenitors (Park, J. E. et al. A cell atlas of human thymic development defines T cell repertoire formation. Science 367, doi:10.1126/science.aay3224 (2020)). The observed prevalence of these TCRV segments in the data presented herein is consistent with the fact that the PSC-derived progenitors that were sequenced contain a higher proportion of DN cells compared to what would be found in the thymus, or in the CB samples we sequenced previously. The mean CDR3 length for PSC-derived cells was slightly shorter than post-natal thymus and closer to the median CDR3 length in T cells differentiated from cord-blood progenitors (FIGS. 23 c and d). This profile is consistent a fetal, rather than post-natal identity.

As such, the methods of the invention illustrate that the T-cells resulting from the methods of the present invention show diversity of T cell receptor range with diverse usage of V and J segments as shown in more mature cells.

### Example 11—Cells Generated by the Method of the Invention are Multipotent and have a Transcriptional Signature that Matches Primary Definitive HSCs from the Carnegie Stage 14/15 Human Aorta-Gonad-Mesonephros (AGM)

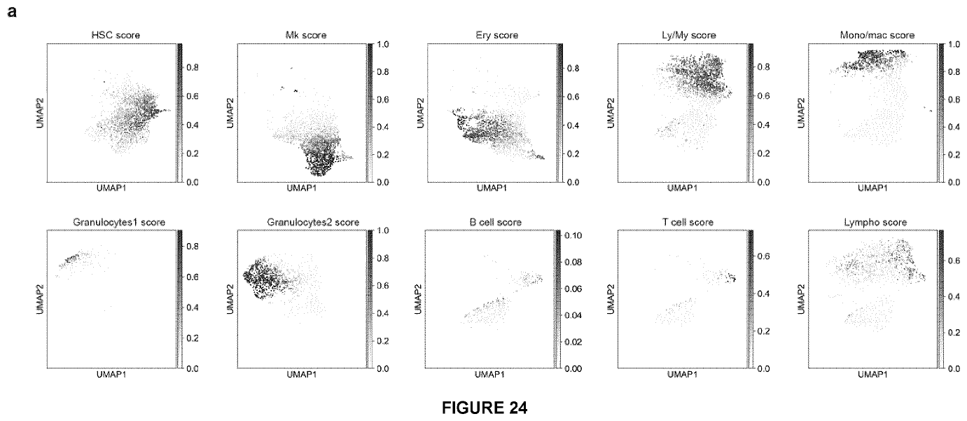

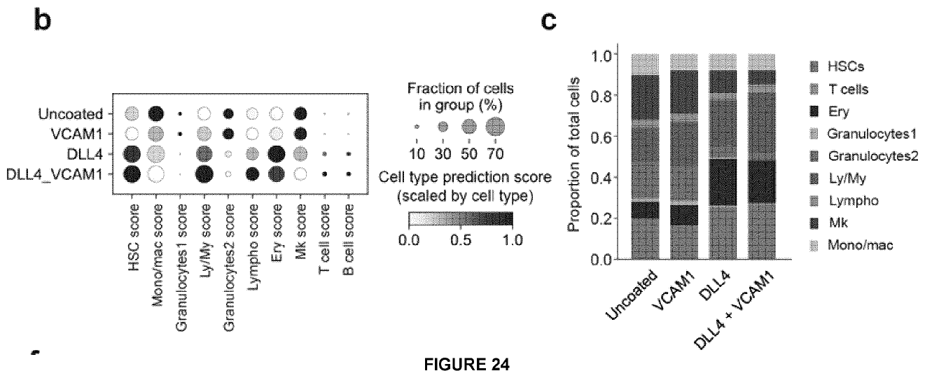

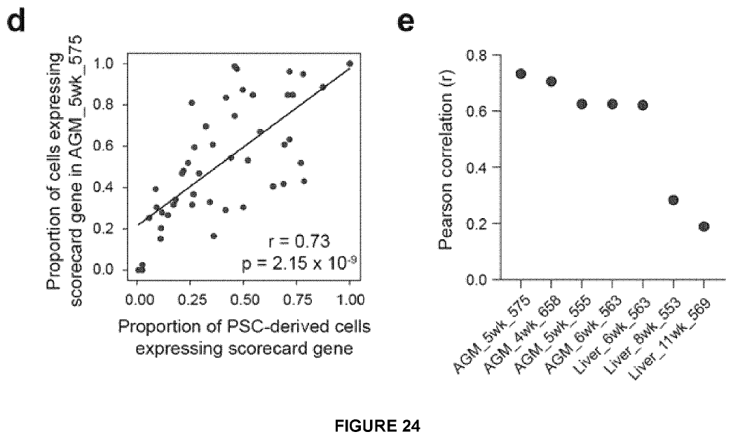

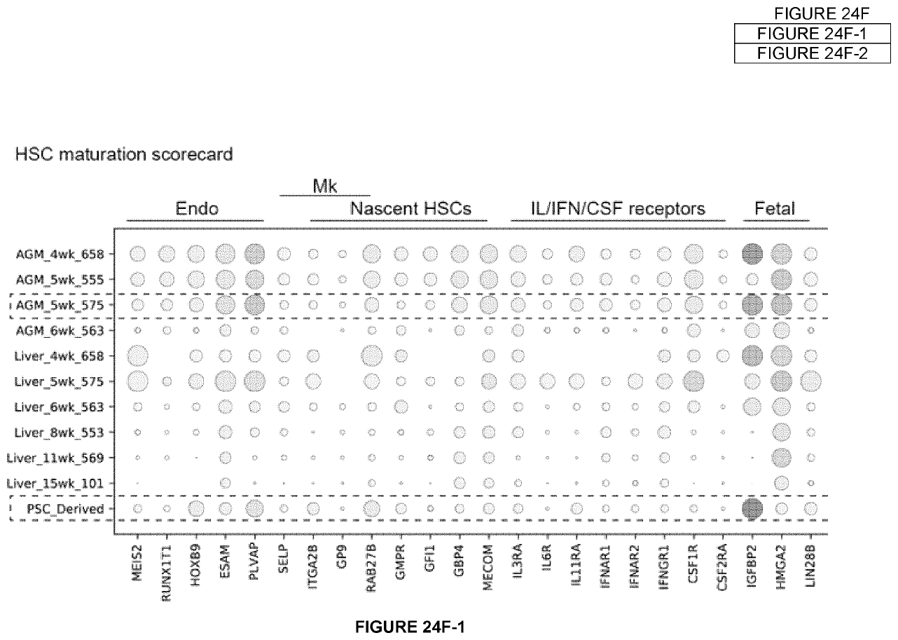

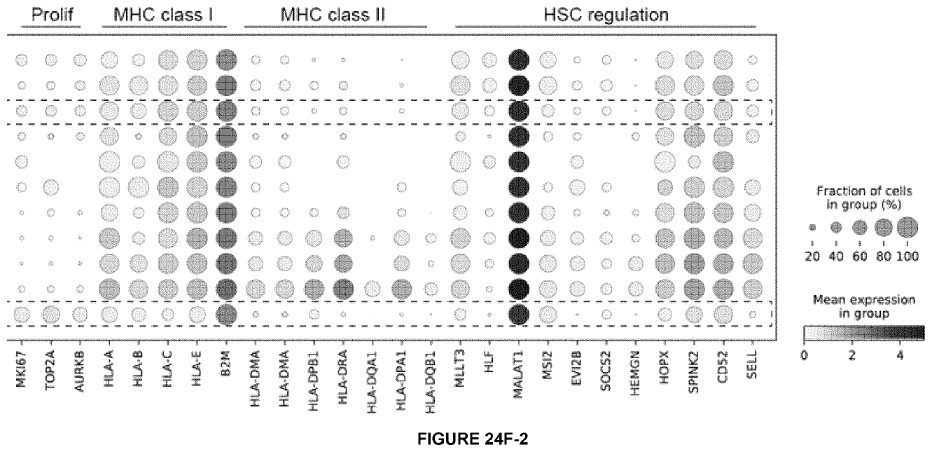

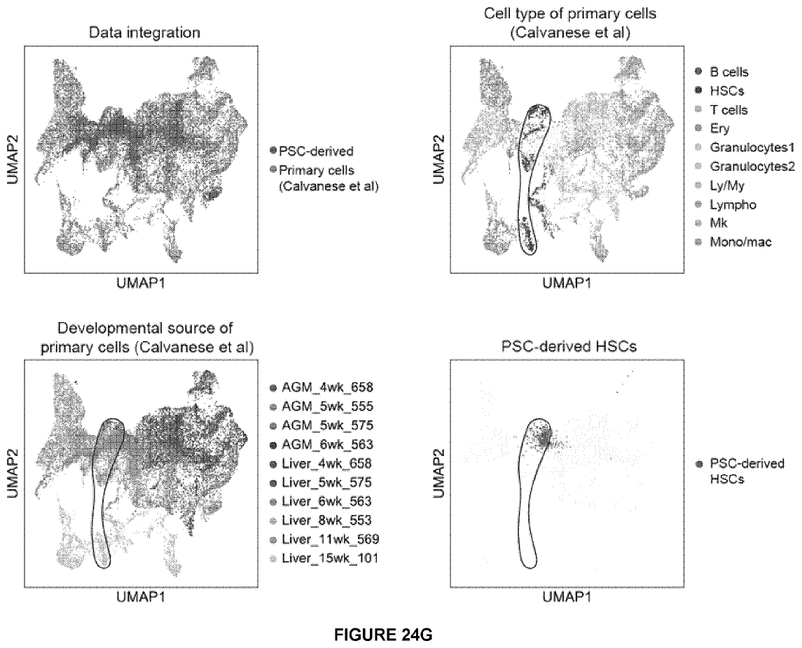

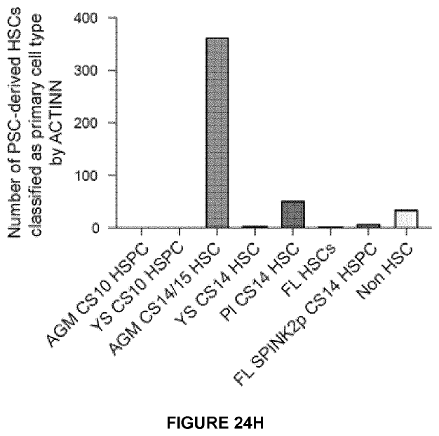

PSC-Derived HSCs Display Strong Transcriptional Correspondence with Primary HSCs from the Human CS14/15 AGM. Results are Shown in FIG. 24.

scRNA sequencing data from PSC-derived HSPC were integrated with a recently published dataset from primary human hematopoietic development Calvanese et al. (Nature, 2022) For PSC-derived cells, a transcriptome-wide cell type prediction score was calculated for each labelled cell type in the Calvanese et al. dataset. UMAPs show cell type prediction scores from primary cells plotted on PSC-derived HSPC (FIG. 24 a.). The FIG. 24 b. Dotplot shows the scores for each primary cell type label (columns) broken down by the coating condition used during EHT to generate the PSC-derived HSPCs of the invention (rows). After classifying PSC-derived cells into primary cell types from Calvanese et al. using a transcriptome-wide anchor-based integration strategy, the frequency of each classified cell type was quantified and plotted them by EHT coating condition (FIG. 24 c.). This analysis corroborates the findings noted previously herein that DLL4 increases HSC and erythroid production while decreasing granulocyte and megakaryocyte (Mk) output. FIG. 24 d, e.) Illustrate the transcriptional identity of PSC-derived cells that were classified as HSCs with primary HSCs from different anatomical locations and developmental time points were compared by comparing expression of genes from an “HSC maturation scorecard” established by Calvanese et al. In FIG. 24 (d) the inventors herein show an example regression comparing PSC-derived HSCs to their most-similar primary counterpart, HSCs from the 5-week AGM. The numbers after each primary cell type label are sample identifiers from Calvanese et al. Note that there are two biological replicates for the 5-week AGM (555 and 575).

The FIG. 24 f.) dotplot shows expression of genes from the HSC maturation score card used for analysis in Figures (d) and (e). Dashed box highlights our PSC-derived HSCs and their most similar primary counterpart, a sample from the 5-week AGM. To further compare our PSC-derived cells to primary cells from Calvanese et al, these two datasets were integrated using the Scanpy ‘ingest’ function and plotted them in a UMAP. The results are shown in FIG. 24 g. This analysis revealed that the PSC-derived HSCs of the present invention occupy a position in the two-dimension projection that overlaps with HSCs from week 5 and 6 AGM and week 6 fetal liver HSCs.

To provide further unbiased, transcriptome-wide characterisation of the PSC-derived HSCs, Automated Cell Type Identification using Neural Networks (ACTINN, Ma and Pellegrini, 2020) was used to classify the cells into their most similar primary counterparts. (See FIG. 24 h.) A training dataset was used from Calvanese et al that comprises multiple hematopoietic cell types from different developmental times and anatomical locations. This neural network model classified the majority of our PSC-derived HSCs as definitive HSCs from the Carnegie stage (CS) 14/15 AGM. The ‘Non HSC’ category contains all cells classified as hemogenic endothelium, arterial endothelium, erythroid/megakaryocyte/mast progenitors or monocyte/macrophage progenitors. Zero PSC-derived HSC cells were classified as granulocytes, erythroid progenitors, granulocytes, mature monocytes/macrophages, T lymphocytes, B lymphocytes or cord-blood HSCs.

### Example 12—PSC Derived Hematopoietic Cells Generated Using the In Vitro Process of the Invention have Multi-Lineage Potential

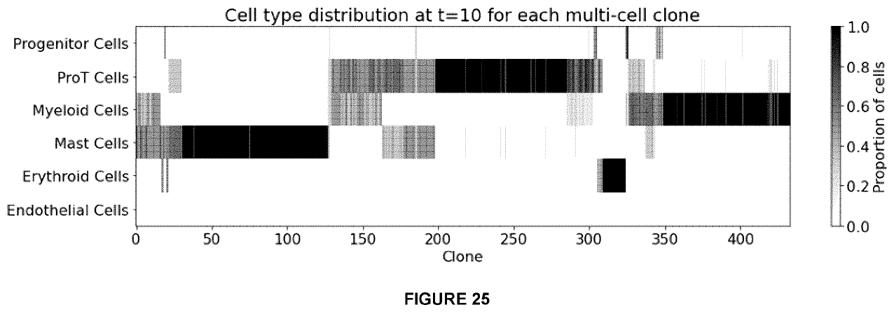

Transcribed lineage barcodes were used to track the output of individual PSC-derived hematopoietic cells. Downstream lineage output was scored by single cell RNA sequencing and unsupervised clustering. Results are shown in FIG. 25. This analysis reveals that individual clones can produce multiple different cell types including multiple clones that are capable of producing at least 3 different cell types.

### References and Modifications

All references listed and disclosed in the specification and Examples, including patents, patent applications, international patent applications and publications are incorporated herein in their entirety by reference.

Although the disclosure has been described with reference to certain specific embodiments, various modifications thereof will be apparent to those skilled in the art. Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. The scope of the claims appended hereto should not be limited by the preferred embodiments set forth in the above description, but should be given the broadest interpretation consistent with the present specification as a whole. The disclosures of all prior art recited herein are incorporated herein by reference in their entirety.

### REFERENCES

Calvanese, V., Capellera-Garcia, S., Ma, F. et al. Mapping human haematopoietic stem cells from hemogenic endothelium to birth. Nature 604, 534-540 (2022). https://doi.org/10.1038/s41586-022-04571-x
Dzierzak, E. et al, Blood Development: Hematopoietic Stem Cell Dependence and Independence, Cell Stem Cell, vol. 22, Issue 5, pp 639-651, (May 3, 2018)
Edgar, J. M., Michaels, Y. S. & Zandstra, P. W. Multi-objective optimization reveals time- and dose-dependent inflammatory cytokine-mediated regulation of human stem cell derived T-cell development. npj Regen Med 7, 11 (2022). https://doi.org/10.1038/s41536-022-00210-1
Goodman and Gilman's The Pharmacological Basis of Therapeutics, 10th Edition, McGraw-Hill (New York) (2001)
Kumar et al (J Immunol 2019; 202:770-776 (pre-published on line Dec. 21, 2018))
Mazo et al, Trends Immunol. 2011 October; 32(10): 493-503).
Montel-Hagen et al, Organoid-Induced Differentiation of Conventional T Cells from Human Pluripotent Stem Cells. Cell Stem Cell. 2019 Mar. 7; 24(3):376-389.e8. doi:10.1016/j.stem.2018.12.011. Epub 2019 Jan. 17. PMID: 30661959; PMCID: PMC6687310.
Ng, E. S. et al. Differentiation of human embryonic stem cells to HOXA(+) hemogenic vasculature that resembles the aorta-gonad-mesonephros. Nat Biotechnol 34, 1168-1179, doi:10.1038/nbt.3702 (2016)
Park, J. E. et al. A cell atlas of human thymic development defines T cell repertoire formation. Science 367, doi:10.1126/science.aay3224 (2020)
Sturgeon, C. M., Ditadi, A., Awong, G., Kennedy, M. & Keller, G. Wnt signaling controls the specification of definitive and primitive hematopoiesis from human pluripotent stem cells. Nat Biotechnol 32, 554-561, doi:10.1038/nbt.2915 (2014).
Sugimura, R. et al. Haematopoietic stem and progenitor cells from human pluripotent stem cells. Nature 545, 432-438, doi:10.1038/nature22370 (2017)
Trotman-Grant, A. C., Mohtashami, M., De Sousa Casal, J. et al. DL4-μbeads induce T cell lineage differentiation from stem cells in a stromal cell-free system. Nat Commun 12, 5023 (2021). https://doi.org/10.1038/s41467-021-25245-8
Uenishi, G. I. et al. NOTCH signaling specifies arterial-type definitive hemogenic endothelium from human pluripotent stem cells. Nat Commun 9, 1828, doi:10.1038/s41467-018-04134-7 (2018)
Ungrin, M. D., Joshi, C., Nica, A., Bauwens, C. & Zandstra, P. W. Reproducible, ultra high-throughput formation of multicellular organization from single cell suspension-derived human embryonic stem cell aggregates. PLoS One 3, e1565, doi:10.1371/journal.pone.0001565 (2008).
The Merck Manual of Diagnosis and Therapy, 19th Edition, published by Merck Sharp & Dohme Corp., 2011 (ISBN 978-0-911910-19-3)
The Encyclopedia of Molecular Cell Biology and Molecular Medicine, published by Blackwell Science Ltd., 1999-2012 (ISBN 9783527600908)
Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006
Janeway's Immunobiology, Kenneth Murphy, Allan Mowat, Casey Weaver (eds.), Taylor & Francis Limited, 2014 (ISBN 0815345305, 9780815345305)
Lewin's Genes XI, published by Jones & Bartlett Publishers, 2014 (ISBN-1449659055)
Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414)
Davis et al., Basic Methods in Molecular Biology, Elsevier Science Publishing, Inc., New York, USA (2012) (ISBN 044460149X)
Laboratory Methods in Enzymology: DNA, Jon Lorsch (ed.) Elsevier, 2013 (ISBN 0124199542)
Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005;
Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737)
U.S. Pat. No. 10,858,628 granted Dec. 8, 2020 (Bahram Valamehr et al.)
U.S. Pat. No. 10,947,502 granted Mar. 16, 2021 (Maksym A. Vodyanyk et al.)
WO/2021/032855 A1 published Feb. 25, 2021 (Yang Cheng Tao et al)
WO2021092699 (PCT/CA2017/050428) published Oct. 12, 2018 (Peter Zandstra et al.)
U.S. Pat. No. 7,947,266 granted May 24, 2011 (Stan Gronthos et al.) published
U.S. Pat. No. 10,220,057 granted Mar. 5, 2019 (Paul Simmons et al.)

1. A method for producing and enhancing the production of blood progenitor cells comprising an endothelial to hematopoietic transition step comprising:
culturing hemogenic endothelial cells under conditions that commit and direct the cells to differentiate into blood progenitor cells (hematopoietic stem cells and progenitor cells), wherein the conditions comprise culturing the hemogenic endothelial cells in a media formulated to promote endothelial to hematopoietic transition (EHT) while enhancing Notch signaling pathway activation,
obtaining a cell culture comprising blood progenitor cells and optionally isolating and selecting for said cells.

2. The method of claim 1, wherein enhancing the Notch signaling pathway activation comprises culturing the cells on a surface functionalised with ligands that enhance activation of the Notch signaling pathway.

3. The method of claim 2 wherein the ligands are adsorbed or immobilized on the surface.

4. The method of any one of claim 2 or 3 wherein the surface functionalised with ligands is selected from: a two dimensional tissue culture surface; a tissue culture plate; the surface of beads; the surface of hydrogels; manufactured or human made surface; and other suitable surfaces.

5. The method of any one of claims 2-4 wherein the surface is functionalised with ligands comprising a Notch ligand and an integrin ligand.

6. The method of claim 5, wherein the integrin ligand is a vascular cell adhesion ligand.

7. The method of claim 6, wherein the integrin ligand is VCAM-1.

8. The method of any one of claims 2-7, wherein the Notch ligand is DLL4.

9. The method of any one of claims 1-8 wherein the method further comprises:
a. a blood induction step prior to the endothelial to hematopoietic transition step comprising:
i. optionally culturing pluripotent stem cells under conditions wherein the pluripotent stem cells aggregate into 3-dimensional multi-cellular structures; and
ii. subjecting the pluripotent stem cells to staged media formulations that direct the cells to commit to mesoderm and subsequently hemogenic endothelial cells wherein optionally some of the hemogenic endothelial cells are aggregated; and
b. wherein the endothelial to hematopoietic transition step optionally comprises prior to the culturing of the hemogenic endothelial cells, dissociating aggregated cells, and optionally enriching the CD34+ cell population which comprises hemogenic endothelial cells.

10. The method of claim 9 wherein dissociating aggregated cells in step b of claim 9 comprises culturing the cells under conditions that dissociate or promote dissociation of the cells.

11. The method of any one of claim 9 or 10 wherein the pluripotent stem cells in step a of claim 9 are aggregated naturally and/or through chemical induction and/or by mechanical or physical means (such as agitation, centrifugation or stirring).

12. The method of any one of claims 9-11 wherein the 3-dimensional multi-cellular structures are 2 or more cells, 5 or more cells, or 10-1000 cell structures.

13. The method of any one of claims 1-12 wherein the blood progenitor cells are capable of lymphoid specification (lymphopoiesis), differentiation into progenitor T cells and T cell differentiation.

14. The method of any one of claims 1-13 wherein the blood progenitor cells are isolated using genetic expression and/or cell surface markers that are characteristic of said cells.

15. The method of any one of claims 1-14 wherein the blood progenitor cells produced or obtainable by the method are characterised in that they express the cell surface markers: SPN, PTPRC, HLF and THY1.

16. The method of any one of claims 1-15 wherein the blood progenitor cells are characterised in that they express the cell surface markers: HLF, THY1, SPN, ERG, HOXA9, HOXA10, LCOR, RUNX1 and SPI1.

17. The method of any one of claims 1-16 wherein the blood progenitor cells produced by the invention can differentiate to become myeloid progenitors, mast cell progenitors, lymphoid progenitors and erythroid progenitors.

18. The method of claim 9 wherein the staged media for culturing the pluripotent stem cells from day 0 to day 7 comprises with an acceptable base media:

| Day | Day | Day | Day | Day | Day | Day | Day |
|---|---|---|---|---|---|---|---|
| 0 | 1 | 1.75 | 3 | 4 | 5 | 6 | 7 |

Ascorbic acid (50 ug/mL)
1-thioglycerol (0.04 ul/mL)
Transferrin (150 ug/ml)
BMP4 (10 ng/mL)
Y-27632
(5 uM)
bFGF (5 ng/ml)
SB-431542 (6 uM)
CHIR-99021 (3 uM)
VEGF(15 ng/mL)
II-6 (10 ng/mL)
IL-11 (5 ng/mL)
EPO (2 U/mL)
IGF-I (25 ng/mL)
SCF (25 ng/mL)

19. The method of any one of claims 1-18 wherein the media formulation to promote endothelial to hematopoietic transition (EHT) comprises the following media in a suitable base media:

| | |
|---|---|
| Concentration Range | Agent |
| | | |
|---|---|---|
| 1-5 | uL/mL | Activation of cellular proliferation |
| 25-100 | ug/mL | Antioxidant, citrriline/NOS |
| | | agonist |
| 50-200 | ug/mL | Mediates iron updake |
| 1-10 | ng/mL | Hematopoeitic growth factor |
| 1-10 | ng/mL | Synergises with IL03 to shorten |
| | | cell cylce |
| 10-50 | ng/mL | Synergises with IL-3 to shorten |
| | | cell cycle |
| 0-10 | ng/mL | Optionally an agent that |
| | | stimulates HSC proliferation |
| 10-50 | ng/mL | Regulates Platelet Production |
| 15-30 | ng/mL | Growth Hormone |
| 50-100 | ng/mL | Promotes blood cell survival, |
| | | proliferation and differentiation |
| 10-30 | ng/mL | Induces profileration and |
| | | differentiation |
| 3-10 | uM | Limits cell death |
| 5-20 | ng/mL | Supports HSC development |
| 5-20 | ng/mL | Stimulates HSC proliferation |

20. The method of claim 19 wherein the media comprises:

| Reagent | Concentration Range | |
|---|---|---|
| 1-thioglycerol [MTG] (Sigma)* | 1-5 | uL/mL |
| Ascorbic Acid (Sigma) | 25-100 | ug/mL |
| Transferrin (Roche) | 50-200 | ug/mL |
| bFGF | 1-10 | ng/mL |
| VEGF | 1-10 | ng/mL |
| IL-6 (dilute stock 1/10) | 10-50 | ng/mL |
| IL-11 | 0-10 | ng/mL |
| TPO | 10-50 | ng/mL |
| IGF-I | 15-30 | ng/mL |
| SCF | 50-100 | ng/mL |
| IL-3 (dilute stock 1/10) | 10-30 | ng/mL |
| ROCKi | 3-10 | uM |
| BMP4 | 5-20 | ng/mL |
| FLT3L (dilute stock 1/10) | 5-20 | ng/mL |

21. The method of claim 20, wherein the media comprises:

| Reagent | Concentration Used | |
|---|---|---|
| 1-thioglycerol [MTG] (Sigma)* | 3 | μl/ml |
| Ascorbic Acid (Sigma) | 50 | μg/ml |
| Transferrin (Roche) | 150 | μg/ml |
| bFGF | 5 | ng/mL |
| VEGF | 5 | ng/mL |
| IL-6 (dilute stock 1/10) | 10 | ng/mL |
| IL-11 | 5 | ng/mL |
| TPO | 30 | ng/mL |
| IGF-I | 25 | ng/mL |
| SCF | 50 | ng/mL |
| IL-3 (dilute stock 1/10) | 10 | ng/ml |
| ROCKi | 10 | nM |
| BMP4 | 10 | ng/ml |
| FLT3L (dilute stock 1/10) | 10 | ng/ml |

22. The method of claim 19-21 wherein the media further comprises one or more of: EPO, Angiotensin II and Losartan Potassium.

23. The method of claim 22 wherein the media comprises one or more of: EPO at a concentration of 2 U/ml, Angiotensin II at a concentration of 10 ug/ml and Losartan Potassium at a concentration of 100 uM may be present in the media.

24. The method of any one of claims 1-23 wherein the blood progenitor cells are isolated by selecting cells comprising CD34+ and CD43+ or CD43+ cell surface markers.

25. Isolated hematopoietic progenitor cells (HPCs) produced or obtainable by the method of any one of claims 1-24.

26. A method for producing T cell progenitor cells comprising culturing the blood progenitor cells produced using the method of any one of claims 1-24 in media and under culture conditions designed to promote and/or that promote lymphoid specification (lymphopoiesis), differentiation into progenitor T cells and T cell differentiation.

27. The method of claim 26, wherein the media is a non-xenogenic, feeder-free and serum free defined culture media,

28. The method of claim 27 wherein the media is in a suitable base media and comprises:

| Reagent | Concentration | |
|---|---|---|
| Ascorbic acid | 60 | uM |
| 2-Mercaptoethanol | 24 | uM |
| SCF | 0.02 | μg/mL |
| Flt3L | 0.02 | μg/mL |
| TPO | 0.02 | μg/mL |
| IL-7 | 0.02 | μg/ml |
| IL-3 | 0.01 | μg/mL |
| TNFa | 0.005 | μg/mL |

29. The method of claim 28, wherein the suitable base media comprises Iscove's Modified Dulbecco's Medium with 20% BIT 9500 Serum Substitute or B27 supplement, and 0.05% human Low-Density Lipoproteins.

30. The method of any one of claims 26-29 wherein media and culture conditions to promote lymphoid specification and differentiation into progenitor T cells and T cell differentiation comprises culturing the blood progenitor cells on a surface functionalised with ligands that activate or enhance activation of T cell development.

31. The method of claim 30 wherein the ligands are adsorbed or immobilized on the surface.

32. The method of any one of claim 30 or 31 wherein the surface functionalised with ligands is selected from: a two dimensional tissue culture surface; a tissue culture plate; the surface of beads; the surface of hydrogels; manufactured or human made surface; and other suitable surfaces.

33. The method of any one of claims 30-32 wherein the ligands that activate or enhance activation of T cell development are ligands that enhance activation of the Notch signaling pathway.

34. The method of claim 33 wherein the surface is functionalised with ligands comprising a Notch ligand and an integrin ligand.

35. The method of claim 34, wherein the integrin ligand is a vascular cell adhesion ligand.

36. The method of claim 35, wherein the integrin ligand is VCAM-1.

37. The method of any one of claims 34-36, wherein the Notch ligand is DLL4.

38. The isolated T-cell progenitor cells produced or obtainable by the method of any one of claims 26-37 where the cells are isolated progenitor T cells expressing CD7, BCL11B, IGLL1 and CXCR4.

39. The method of claim 26 further comprising a step or steps to differentiate the T cell progenitor cells to mature T cells by culturing them in media or stage media formulated to support T cell development on ligands designed to activate and/or to enhance the activation of the Notch signalling pathway, wherein the ligands are a Notch ligand and an integrin ligand, wherein the ligands are adsorbed or immobilized on or in a surface.

40. The method of claim 39 for producing early and later stage progenitor T cells and mature T cells.

41. The method of any one of claims 1-24 or 26-37 or 39 done ex vivo.

42. Isolated progenitor and later stage T cells obtainable from any one of the methods of claims 26-37 or 39-41.

43. An isolated T cell or isolated T cell population that express αβTCR, CD3 and CD8α and CD8β produced or obtainable using the method of any one of claims 26-37 or 39-42.

44. A population of T cells obtained by the method of any one of claims 26-37 or 39-41, wherein the cells comprise multiple different recombined TCR sequences with similar diversity as naturally occurring T cell populations in vivo.

45. A use of the T cells produced by the method of any one of claims 26-37 or 39-41 or the isolated cells of 38 or 42 or 43 or the population of cells of claim 44 for immunotherapy.

46. The use of claim 45 for use in immunotherapy selected from the following therapies: CAR-T, engineered TCR T cell, T-regulatory cell, genetic modification therapy and other uses.

47. A use of the blood progenitor cells obtained from the method of any one of claims 1-24 or the cells of claim 25 in immunotherapy.

48. The use of claim 47 wherein the immunotherapy comprises administering the cells in a patient in need of a hematopoietic stem cell protocol or a bone marrow transplant.

49. The use of the cells produced by the methods of any one of claims 1-24 and 26-37 or 39-41 or the isolated cells of 38 or 42 or 43 or the population of cells of claim 44 in the manufacture of a medicament for immunotherapy treatment of a patient in need thereof.

50. A method for producing blood progenitor cells comprising:
a. a blood induction step comprising:
i. optionally culturing cells under conditions wherein pluripotent stem cells aggregate into 3-dimensional multi-cellular structures; and
ii. subjecting pluripotent stem cells to staged media formulations that direct the cells to commit to mesoderm and subsequently hemogenic endothelial cells wherein optionally some of the pluripotent stem cells are aggregated pluripotent stem cells;
b. an endothelial to hematopoietic transition step comprising:
i. optionally dissociating aggregated cells, and optionally enriching the CD34+ cell population which comprises hemogenic endothelial cells; and
ii. culturing the hemogenic endothelial cells under conditions that commit and direct the cells to differentiate into hematopoietic stem and progenitor cells, wherein the conditions comprise using a media formulation designed to promote endothelial to hematopoietic transition (EHT) while being cultured on a surface functionalised with ligands designed to activate and/or enhance the activation of the Notch signaling pathway, wherein the ligands comprise a Notch signaling ligand and an integrin ligand,
obtaining a cell culture comprising blood progenitor cells and optionally isolating and selecting for said cells.

51. A method of any one of claims 1-24 or 50 wherein the blood progenitor cells produced from the hemogenic endothelial cells are definitive blood progenitor cells.

52. A method of any one of claims 1-24 or 50 or 51 wherein the blood progenitor cells produced express a molecular signature consistent with primary definitive HSCs.

53. The method of claim 52 wherein the blood progenitor cells express a molecular signature comprising the expression of both HLF and HOXA9 and optionally one or both of RAB27B and IGFBP2.

54. A method of any one of claims 1-24 or 50-53 wherein the blood progenitor cells produced have multilineage developmental capacity.

55. The method of any one of claims 1-24 or 50-54, wherein the blood progenitor cells produced can develop into differentiated blood cells.

56. The method of claim 55 wherein the differentiated blood cells are selected from the group consisting of erythroid cells, macrophages, mast cells, B cells, T cells, megakaryocytes, granulocytes, neutrophils, natural killer (NK) cells, and eosinophils.


SEQUENCE LISTING (5 sequences)

SEQ ID NO: 1 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 2 - protein, 6 aa, Homo sapiens
QIDSPL

SEQ ID NO: 3 - protein, 8 aa, Homo sapiens
TQIDSPLN

SEQ ID NO: 4 - protein, 739 aa, Mus musculus
MPVKMVAVLGASTVLWILFAVSQAFKIEISPEYKTIAQIGDSMALTCSTTGCESPLFSWRTQIDSPLNAKVRTEGSKSVLTMEPVSFENEHSYLCTATCGSGKLERSIHVDIYSFPKDPEIQFSGPLEVGKPVTVKCLAPDIYPVYRLEIDLFKGDQLMNRQEFSSEEMTKSLETKSLEVTFTPVIEDIGKALVCRAKLHIDQIDSTLKERETVKELQVYISPRNTTISVHPSTRLQEGGAVTMTCSSEGLPAPEIFWGRKLDNEVLQLLSGNATLTLIAMRMEDSGVYVCEGVNLIGRDKAEVELVVQEKPFIVDISPGSQVAAQVGDSVVLTCAAIGCDSPSFSWRTQTDSPLNGVVRNEGAKSTLVLSSVGFEDEHSYLCAVTCLQRTLEKRTQVEVYSFPEDPVIKMSGPLVHGRPVTVNCTVPNVYPFDHLEIELLKGETTLMKKYFLEEMGIKSLETKILETTFIPTIEDTGKSLVCLARLHSGEMESEPKQRQSVQPLYVNVAPKETTIWVSPSPILEEGSPVNLTCSSDGIPAPKILWSRQLNNGELQPLSENTTLTFMSTKRDDSGIYVCEGINEAGISRKSVELIIQVSPKDIQLTVFPSKSVKEGDTVIISCTCGNVPETWIILKKKAKTGDMVLKSVDGSYTIRQAQLQDAGIYECESKTEVGSQLRSLTLDVKGKEHNKDYFSPELLALYCASSLVIPAIGMIVYFARKANMKGSYSLVEAQKSKV

SEQ ID NO: 5 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV